(19) United States
(12) Patent Application Publication
(10) Pub. No.: US 2023/0099024 A1
(43) Pub. Date: Mar. 30, 2023

(54) SYSTEM AND METHOD FOR CONTROLLING SUPERSATURATED OXYGEN THERAPY BASED ON PATIENT PARAMETER FEEDBACK

(71) Applicant: Zoll Circulation, Inc., San Jose, CA (US)

(72) Inventors: Gary A. Freeman, Waltham, MA (US); Paolo Giacometti, North Grafton, MA (US); Joshua W. Lampe, Groton, MA (US); Ulrich Herken, Medford, MA (US); Stephen Elwood Myrick, Tustin, CA (US); Jeffrey Lance Creech, Los Angeles, CA (US)

(21) Appl. No.: 17/802,880

(22) PCT Filed: Mar. 30, 2021
(86) PCT No.: PCT/US2021/024992

§ 371 Date: Aug. 26, 2022

Related U.S. Application Data
(60) Provisional application No. 63/003,210, filed on Mar. 31, 2020.

(51) Int. Cl. A61M 1/32

(52) U.S. Cl. CPC A61M 1/32; A61M 2205/502; A61M 2205/52; A61M 2205/18

(57) ABSTRACT

The present disclosure provides systems and methods for controlling gas enrichment therapy. One or more sensors is used to measure one or more physiological parameters, e.g., blood or tissue oxygen parameters, of the patient. A processor is used to generate based on the measured parameters an alert through a user interface indicating a value or level of the measured physiological parameter, which is indicative of an effectiveness of the gas enrichment therapy.

### CLAIM OF PRIORITY

This application claims priority under 35 U.S.C. § 119(e) U.S. Patent Application Ser. No. 63/003,210, filed on Mar. 31, 2020, the entire contents of which are incorporated herein by reference.

### TECHNICAL FIELD

The present application relates generally to the field of gas enrichment therapy, or supersaturated oxygen or gas therapy systems.

### BACKGROUND

Blockage of oxygenated blood flow can cause a heart attack. During such an occurrence, tiny heart capillaries can swell further restricting blood flow in a manner that can cause damage to the heart muscle or an infarction. Supersaturated oxygen therapy systems infuse superoxygenated blood into a patient's coronary artery to improve microvascular flow to restore heart tissue to normal oxygen level. Superoxygenated blood can be provided via a catheter and can help reduce infarct size.

### SUMMARY

The present disclosure provides systems and methods for monitoring, analyzing, delivering and/or controlling gas enrichment therapy or supersaturated oxygen or gas therapy. One or more sensors may be used to measure one or more physiological parameters, e.g., blood or tissue oxygen parameters, of the patient. A processor may be used to generate based on the measured parameters an alert through a user interface indicating a value or level of the measured physiological parameter, which is indicative of an effectiveness of the gas enrichment therapy or supersaturated gas therapy. According to a first example, systems for monitoring, analyzing, delivering and/or controlling supersaturated oxygen or gas therapy are disclosed. The systems include a gas enrichment system configured to enrich a liquid with gas to form a gas enriched liquid and to mix the gas enriched liquid with blood, e.g., arterial blood, which may form gas enriched blood. The systems include a plurality of fluid conduits fluidly coupled to the gas enrichment system. At least one conduit of the plurality of fluid conduits is configured for flow of the blood from the patient to the gas enrichment system, and at least one conduit of the plurality of conduits is configured for flow of gas-enriched blood from the gas enrichment system to the patient. The systems include a blood pump coupled to at least one conduit of the plurality of fluid conduits, for pumping blood to and from the gas enrichment system and the patient. The systems include at least one sensor configured to measure one or more blood oxygen parameters. The systems include a user interface configured to receive user input and emit at least one of a visual alert and an audible alert and a controller. The controller includes a processor, a memory, and associated circuitry communicatively coupled to the at least one sensor and the user interface. The controller or processor is configured to receive one or more signals corresponding to a measured value of the one or more blood oxygen parameters from the at least one sensor and generate, based on the measured value, an alert through the user interface indicative of the measured value of the blood oxygen parameter, which is indicative of an effectiveness of the gas enrichment therapy or supersaturated oxygen therapy.

In certain implementations, the gas enrichment system is configured to enrich a liquid with oxygen to form an oxygen enriched liquid to be mixed with blood.

In certain implementations, the one or more blood oxygen parameters comprises arterial pO₂.

In certain implementations, the at least one sensor comprises a Clark electrode for measuring the pO₂.

In certain implementations, the one or more blood oxygen parameters comprises arterial SO₂.

In certain implementations, the processor compares the measured value for pO₂ to a preprogrammed target range for pO₂ of 760-1200 mmHg or 760-1500 mmHg.

In certain implementations, the processor controls delivery of gas-enriched blood to the patient based on the comparison.

In certain implementations, the one or more blood oxygen parameters is arterial SO₂ and the processor compares the measured value for SO₂ to an accepted normal range for arterial SO₂ is 90-100 percent.

In certain implementations, the one or more blood oxygen parameters is arterial pO₂ and the processor compares the measured value for pO₂ to an accepted normal range for arterial pO₂ is 75-100 mmHg or 75-110 mmHg.

In certain implementations, the gas-enrichment system comprises a cartridge.

In certain implementations, the cartridge has three chambers.

According to a second example, systems for monitoring, analyzing, delivering and/or controlling gas enrichment therapy or supersaturated oxygen or gas therapy in a patient are disclosed. The systems include a gas enrichment system configured to enrich a fluid or liquid with gas to form a gas-enriched fluid or liquid and to mix the gas enriched fluid or liquid with blood e.g., to form gas enriched blood. The systems include a plurality of fluid conduits fluidly coupled to the gas enrichment system. At least one conduit of the plurality of fluid conduits is configured for flow of the blood from the patient to the gas enrichment system, and at least one conduit of the plurality of conduits is configured for flow of gas-enriched blood from the gas enrichment system to the patient. The systems include a blood pump coupled to at least one conduit of the plurality of fluid conduits, for pumping blood to and from the gas enrichment system and the patient. The systems includes a catheter coupled to the at least one conduit configured for flow of gas enriched blood to the patient. The catheter includes one or more internal electrodes coupled thereto. The system includes a plurality of external electrodes configured to be coupled to an external surface of a patient. The systems include a user interface configured to receive user input and emit at least one of a visual alert and an audible alert. The systems include a controller including a processor, a memory, and associated circuitry communicatively coupled to the one or more internal electrodes of the catheter, a plurality of external electrodes and a user interface. The controller or processor is configured to receive a plurality of signals corresponding to measured impedance values from a tissue area between the one or more internal electrodes and plurality of external electrodes, generate an impedance tomographic map based at least in part on the measured impedance values, and provide, through the user interface, information regarding blood perfusion in the tissue area based on the tomographic map, which information is indicative of an effectiveness of the gas enrichment therapy or supersaturated oxygen therapy.

In certain implementations, the gas enrichment system is configured to enrich a liquid with oxygen to form an oxygen enriched liquid to be mixed with blood.

In certain implementations, the tissue perfusion information based on the tomographic map comprises increased blood perfusion and reduced infarct, which is represented by map zones having low impedance values.

In certain implementations, the tissue area includes an infarct, and the processor is configured to compare the tomographic map of measured impedance values in the tissue area to a baseline tomographic map of measured impedance values in the tissue area to determine changes in blood perfusion or infarct size in the patient.

In certain implementations, the processor is configured to tag map zones and analyze a change in tissue impedance for the tagged map zone over a period of time.

In certain implementations, the processor is configured to calculate an average tissue impedance for the tagged map zone over a period of time.

In certain implementations, the one or more catheter electrodes comprises a bipolar ECG electrode.

In certain implementations, the processor is configured to cause the gas enrichment system to increase a level of O₂ saturation in the blood based on the tissue perfusion information.

In certain implementations, the processor is configured to cause the pump to increase a flowrate of oxygen-enriched blood to the patient based on the tissue perfusion information.

In certain implementations, the processor is configured to overlay the tomography map on an MRI or CT image of the tissue area showing an infarct zone, and the processor is configured to calculate the average impedance in the infarct zone.

According to a third example, systems for monitoring, analyzing, delivering and/or controlling gas enrichment therapy or supersaturated oxygen or gas therapy in a patient are disclosed. The systems include a gas enrichment system configured to enrich a fluid or liquid with gas to form a gas-enriched fluid or liquid and to mix the gas enriched fluid or liquid with blood e.g., to form gas enriched blood. The systems include a plurality of fluid conduits fluidly coupled to the gas enrichment system, at least one conduit of the plurality of fluid conduits configured for flow of the blood from the patient to the gas enrichment system, and at least one conduit of the plurality of conduits configured for flow of gas-enriched blood from the gas enrichment system to the patient. The systems include a blood pump coupled to at least one conduit of the plurality of fluid conduits, for pumping blood to and from the gas enrichment system and the patient. The systems include a nuclear magnetic resonance probe configured to measure a resonance signal of a target molecule in a target tissue. The systems include a user interface configured to receive user input and emit at least one of a visual alert and an audible alert. The systems include a controller including a processor, a memory, and associated circuitry communicatively coupled to the magnetic resonance imaging probe and the user interface. The controller or processor is configured to receive one or more signals corresponding to a level of the target molecule in the target tissue based on the measured resonance signal of the molecule from the nuclear magnetic resonance imaging probe, and generate, based on the measured value, an alert through the user interface indicating the level of the target molecule in the target tissue, which is indicative of an effectiveness of the gas enrichment therapy or supersaturated oxygen therapy.

In certain implementations, the gas enrichment system is configured to enrich a liquid with oxygen to form an oxygen enriched liquid to be mixed with blood.

In certain implementations, the target molecule in the target tissue comprises oxygen in blood

In certain implementations, the level of oxygen in blood refers to SO₂ in blood.

In certain implementations, the target molecule in the target tissue comprises high-energy phosphate in blood, wherein a level of high-energy phosphate in blood is indicative of the tissue's metabolic state.

In certain implementations, the processor is configured to generate a magnetic resonance image of the target tissue and analyze the image to detect the presence of the target molecule in the target tissue.

In certain implementations, the magnetic resonance imaging probe comprises a magnetic coil wound around a peripheral portion of the catheter, the catheter coupled to the at least one conduit configured for flow of gas-enriched blood to the patient.

In certain implementations, the magnetic resonance imaging probe comprises a magnetic resonance imaging receiver on the end of a catheter, the catheter coupled to the at least one conduit configured for flow of gas-enriched blood to the patient

According to a fourth example, systems for monitoring, analyzing, delivering and/or controlling gas enrichment therapy or supersaturated oxygen or gas therapy in a patient are disclosed. The systems include a gas enrichment system configured to enrich a fluid or liquid with oxygen to form an oxygen enriched fluid or liquid and to mix the oxygen enriched fluid or liquid with blood e.g., to form gas enriched blood. The systems include a plurality of fluid conduits fluidly coupled to the gas enrichment system, at least one conduit of the plurality of fluid conduits configured for flow of the blood from the patient to the gas enrichment system, and at least one conduit of the plurality of conduits configured for flow of gas-enriched blood from the gas enrichment system to the patient. The systems include a blood pump coupled to at least one conduit of the plurality of fluid conduits, for pumping blood to and from the gas enrichment system and the patient. The systems include an O₂ fluorescence probe comprising one or more sensor molecules. The systems include a user interface configured to receive user input and emit at least one of a visual alert and an audible alert. The systems include a controller including a processor, a memory, and associated circuitry communicatively coupled to the O₂ fluorescence probe and the user interface. The controller or processor is configured to receive one or more signals corresponding to a measured fluorescence of the sensor molecule on the O₂ fluorescence probe, determine SO₂ in blood based on the one or more signals, and generate, based on the determined SO₂, an alert through the user interface indicating an effectiveness of the gas enrichment therapy or supersaturated oxygen therapy.

In certain implementations, the O₂ fluorescence probe comprises a catheter.

In certain implementations, the O₂ fluorescence probe comprises a sensor molecule coated onto an end of a fiber optic cable.

In certain implementations, the sensor molecule comprises a fluorophore or phosphor.

In certain implementations, the processor is configured to measure fluorescence signal decay from the sensor molecule due to quenching by O₂, wherein the signal decay time is proportional to SO₂ or pO₂ in the blood.

According to a fifth example, systems for monitoring, analyzing, delivering and/or controlling gas enrichment therapy or supersaturated oxygen or gas therapy in a patient are disclosed. The systems include a gas enrichment system configured to enrich a fluid or liquid with oxygen to form an oxygen enriched fluid or liquid, a pump, a plurality of fluid conduits fluidly coupled to the pump, at least one conduit in the plurality of conduits configured for flow of oxygen-enriched fluid or liquid generated by the gas enrichment system into a patient's blood vessel, a transcutaneous pO₂ probe configured to measure pO₂ in a tissue area, a user interface configured to receive user input and emit at least one of a visual alert and an audible alert; and a controller. The pump may be a blood pump coupled to at least one conduit of the plurality of fluid conduits, for pumping blood to and from the gas enrichment system and the patient. The controller includes a processor, a memory, and associated circuitry communicatively coupled to the transcutaneous pO₂ probe and a user interface. The controller or processor is configured to receive one or more signals corresponding to a measured value of the pO₂ in the tissue area from the transcutaneous pO₂ probe, and generate, based on the measured value, an alert through the user interface indicating an effectiveness of the gas enrichment therapy or supersaturated oxygen therapy.

In certain implementations, the at least one conduit comprises a catheter configured to inject oxygen-enriched saline into the patient's blood vessel.

In certain implementations, the processor controls the delivery of the oxygen-enriched saline into the blood based on the measured pO₂ value.

In certain implementations, the measured value of the pO₂ in the tissue area comprises pO₂ in myocardial tissue.

In certain implementations, the measured value of the pO₂ in the tissue area comprises pO₂ in a coronary blood vessel.

According to a sixth example, systems for monitoring, analyzing, delivering and/or controlling gas enrichment therapy or supersaturated oxygen or gas therapy in a patient are disclosed. The systems include a gas enrichment system configured to enrich a fluid or liquid with gas to form a gas-enriched fluid or liquid and to mix the gas enriched fluid or liquid with blood e.g., to form gas enriched blood, a plurality of fluid conduits fluidly coupled to the gas enrichment system, at least one conduit of the plurality of fluid conduits configured for flow of the blood from the patient to the gas enrichment system, and at least one conduit of the plurality of conduits configured for flow of gas-enriched blood from the gas enrichment system to the patient, a blood pump coupled to at least one conduit of the plurality of fluid conduits, for pumping blood to and from the gas enrichment system and the patient; a photoacoustic imaging light source configured to illuminate a tissue area with a pulse of light, an ultrasonic sensor configured to detect acoustic waves generated by light-absorbing components in the tissue area responsive to illumination by the pulse of light, a user interface configured to receive user input and emit at least one of a visual alert and an audible alert; and a controller. The controller includes a processor, a memory, and associated circuitry communicatively coupled to the photoacoustic imaging probe, the ultrasonic sensor and the user interface. The controller or processor is configured to receive one or more signals corresponding to the detected acoustic waves, generate, based on the detected acoustic waves, an image, and provide, through the user interface, blood oxygenation information about the tissue area based on the image, which information is indicative of an effectiveness of the gas enrichment therapy or supersaturated oxygen therapy.

In certain implementations, the gas enrichment system is configured to enrich a liquid with oxygen to form an oxygen enriched liquid to be mixed with blood.

In certain implementations, the processor controls the delivery of oxygen-enriched blood to the patient based on tissue or blood oxygenation information from the image

In certain implementations, the image is tracked over time to determine a change in blood oxygenation in the tissue area over time.

In certain implementations, the image is tracked over time to determine a presence of or change in blood flow or blood oxygenation in the tissue area over time.

In certain implementations, the photoacoustic imaging light source comprises a fiberoptic cable coupled to a catheter, the catheter configured to deliver the gas-enriched blood to the patient.

In certain implementations, the processor is further configured to generate a tomographic image of the tissue area.

In certain implementations, the photoacoustic imaging light source comprises a laser or pulsed laser diode for generating the pulse of light.

In certain implementations, the blood oxygenation information comprises a change in oxygenated hemoglobin levels represented by a contrast in the image that results from optical absorption properties differing for oxygenated hemoglobin and deoxygenated hemoglobin.

In certain implementations, the photoacoustic imaging light source comprises a light emitting diode for generating the pulse of light.

In certain implementations, the ultrasonic sensor comprises a piezoelectric element.

In certain implementations, the piezoelectric element comprises a linear, piezoelectric, ultrasound transducer array.

In certain implementations, the ultrasonic sensor comprises a Fabry-Perot Interferometer (FPI) element.

In certain implementations, the processor is further configured to raster scan the FPI.

In certain implementations, the pulse of light is in a visible portion of an electromagnetic spectrum.

In certain implementations, the pulse of light is within a near-infrared portion of an electromagnetic spectrum.

In certain implementations, the processor is further configured to generate a two-dimensional image of the tissue area.

In certain implementations, the processor is further configured to generate a three-dimensional image of the tissue area.

According to a seventh example, systems for monitoring, analyzing, delivering and/or controlling gas enrichment therapy or supersaturated oxygen or gas therapy in a patient are disclosed. The systems include a gas enrichment system configured to enrich a liquid with gas to form a gas enriched liquid and to mix the gas enriched liquid with blood, such as arterial blood, e.g., to form gas enriched blood, a blood pump for pumping blood to and from the gas enrichment system and the patient; and a controller. The controller may include a processor, a memory, and associated circuitry for communicatively coupling to at least one sensor configured to measure one or more physiological values. The processor is configured to receive one or more signals corresponding to a measured value of the one or more physiological parameters from the at least one sensor. The controller or processor may be configured to generate, based on the measured value, an alert indicative of the measured value of physiological parameter, which is indicative of an effectiveness of the gas enrichment therapy or supersaturated oxygen therapy. The systems may comprise a user interface. The circuitry of the controller may be communicatively coupled to the user interface. The user interface may be configured to receive user input. The user interface may be configured to emit an alert. The alert may be at least one of a visual alert and an audible alert. The controller or processor may be configured to generate the alert through the user interface. The controller or processor may be configured to control delivery of gas-enriched blood to the patient. The controller or processor may be configured to control delivery of gas-enriched blood to the patient based on the one or more signals or the measured value. The controller or processor may be configured to both generate the alert and control the delivery of gas-enriched blood to the patient. The seventh example may be implemented using any of the implementations described with reference to the first to sixth examples.

In certain implementations, the gas enrichment system is configured to enrich a liquid with oxygen to form an oxygen enriched liquid to be mixed with blood.

In certain implementations, a plurality of fluid conduits are fluidly coupled to the gas enrichment system, where at least one conduit of the plurality of fluid conduits is configured for flow of the blood from the patient to the gas enrichment system, and at least one conduit of the plurality of conduits configured for flow of gas-enriched blood from the gas enrichment system to the patient. The blood pump may be coupled to at least one conduit of the plurality of fluid conduits.

In certain implementations, the at least one sensor comprises a Clark electrode for measuring the pO₂ in blood.

In certain implementations, the systems include the at least one sensor configured to measure the one or more signals corresponding to one or more physiological parameters. The at least one sensor may be provided by one or more of: a catheter coupled to the conduit configured for flow of gas enriched blood to the patient, the catheter comprising one or more internal electrodes coupled thereto; a plurality of external electrodes configured to be coupled to an external surface of a patient; a nuclear magnetic resonance probe configured to measure a resonance signal of a target molecule in a target tissue; an O₂ fluorescence probe comprising one or more sensor molecules; a transcutaneous pO₂ probe configured to measure pO₂ in a tissue area; and an ultrasonic sensor configured to detect acoustic waves generated by light-absorbing components in the tissue area responsive to illumination by a pulse of light.

In certain implementations, the processor compares the measured value for pO₂ to a preprogrammed target range for pO₂ of 760-1200 mmHg or 760-1500 mmHg.

In certain implementations, the processor controls delivery of gas-enriched blood to the patient based on the comparison.

In certain implementations, the processor compares the measured value for SO₂ to an accepted normal range for arterial SO₂ of 90-100 percent.

In certain implementations, the processor compares the measured value for pO₂ to an accepted normal range for arterial pO2, which is 75-100 mmHg.

In certain implementations, the gas-enrichment system comprises a cartridge.

In certain implementations, the cartridge has three chambers.

In certain implementations, the physiological parameter is one or more of: a blood oxygen parameter, which may comprise arterial pO₂ and/or SO₂; arterial blood pressure and an electrical activity of the heart measured (which may be measured by an ECG sensor). The one or more signals may comprise: signals from an ECG sensor measuring electrical activity of the heart; a measured impedance value from a tissue area between a plurality of internal electrodes; a measured impedance value from a tissue area between a plurality of external electrodes; a measured impedance value from a tissue area between one or more internal electrodes and one or more external electrodes; one or more signals corresponding to a level of a target molecule in the target tissue; a measured fluorescence of a sensor molecule on an O₂ fluorescence probe; signals corresponding to a measured value of pO₂ in the tissue area from a transcutaneous pO₂ probe; signals corresponding to detected acoustic waves.

According to a further example, there may be provided a computer implemented method of performing the functions of the controller described previously with respect to any of the first to seventh examples. According to a further example, there may be provided a computer program product, or non-transitory computer readable medium, comprising computer program instructions configured to cause a processor to perform the functions described previously with respect to any of the first to seventh examples.

In a further example, methods for monitoring, analyzing, delivering and/or controlling gas enrichment therapy or supersaturated oxygen or gas therapy in a patient are disclosed. The methods include measuring, via one or more sensors, one or more blood oxygen parameters of the patient transmitting one or more signals to a processor, the one or more signals corresponding to a measured value of the one or more blood oxygen parameters from the at least one sensor; and generating, based on the measured value, an alert through a user interface indicating a measured value of the blood oxygen parameter indicative of an effectiveness of the gas enrichment therapy or supersaturated oxygen therapy.

In certain implementations, measuring includes measuring via a sensor positioned in a catheter.

In certain implementations, measuring includes measuring pO₂ of the blood.

In certain implementations, measuring includes measuring SO₂ of the blood.

In certain implementations, the methods include comparing the measured value for the one or more blood oxygen parameters to an accepted normal range for the one or more blood oxygen parameters in non-ischemic tissue.

In certain implementations, the methods include controlling, via the processor, delivery of gas-enriched blood to the patient based on the comparison of the measured value to the accepted normal range.

In a further example, methods for monitoring, analyzing, delivering and/or controlling gas enrichment therapy or supersaturated oxygen or gas therapy in a patient are disclosed. The methods include measuring, impedance values from a tissue area between the one or more internal catheter electrodes and plurality of external electrodes, generating a tomographic map of the measured impedance values, and providing, through a user interface, tissue perfusion information regarding blood perfusion in the tissue area based on the tomographic map, which information is indicative of an effectiveness of the gas enrichment therapy or supersaturated oxygen therapy.

In certain implementations, the methods include tagging map zones and analyzing a change in tissue impedance for the tagged map zone over a period of time.

In certain implementations, the methods include calculating an average tissue impedance for the tagged map zone over a period of time.

In certain implementations, the methods include causing a gas enrichment system to increase a level of O₂ saturation in the blood based on the tissue perfusion information.

In certain implementations, the methods include causing a pump to increase a flowrate of oxygen-enriched blood to the patient based on the tissue perfusion information.

In certain implementations, the methods include overlaying the tomography map on an MRI or CT image of the tissue area showing an infarct zone, and calculating the average impedance in the infarct zone.

In a further example, methods for monitoring, analyzing, delivering and/or controlling gas enrichment therapy or supersaturated oxygen or gas therapy in a patient are disclosed. The methods include measuring one or more tissue parameters of a resonance of a target molecule in a target tissue using a nuclear magnetic resonance probe. The methods include receiving one or more signals corresponding to a level of a target molecule in a target tissue based on the measured resonance of the molecules from a nuclear magnetic resonance imaging probe. The methods include generating, based on the measured value, an alert through the user interface, the alert indicating the level of the target molecule in the target tissue, which is indicative of an effectiveness of the gas enrichment therapy or supersaturated oxygen therapy.

In certain implementations, the methods include generating a magnetic resonance image of the target tissue and analyze the image to detect the presence of the target molecule in the target tissue.

In a further example, methods for monitoring, analyzing, delivering and/or controlling gas enrichment therapy or supersaturated oxygen or gas therapy in a patient are disclosed. The methods include measuring fluorescence of a sensor molecule on an O₂ fluorescence probe. The methods include receiving one or more signals corresponding to the measured fluorescence of the sensor molecule on the O₂ fluorescence probe. The methods include determining SO₂ in blood based on the one or more signals. The methods include generating, based on the determined SO₂, an alert through the user interface indicating an effectiveness of the gas enrichment therapy or supersaturated oxygen therapy.

In certain implementations, the methods include measuring fluorescence signal decay from the sensor molecule due to quenching by O₂, wherein the signal decay time is proportional to SO₂ in the blood.

In a further example, methods for monitoring, analyzing, delivering and/or controlling gas enrichment therapy or supersaturated oxygen or gas therapy in a patient are disclosed. The methods include measuring pO₂ in a tissue area using a transcutaneous pO₂ probe, receiving one or more signals corresponding to the measured pO₂ in the tissue are from the transcutaneous pO₂ probe, and generating, based on the measured pO₂, an alert through the user interface indicating an effectiveness of the gas enrichment therapy or supersaturated oxygen therapy.

In certain implementations, the methods include controlling a delivery of oxygen-enriched saline into blood based on the measured pO₂ value.

In a further example, methods for monitoring, analyzing, delivering and/or controlling gas enrichment therapy or supersaturated oxygen or gas therapy in a patient are disclosed. The methods include illuminating a tissue area with a pulse of light from a photoacoustic imaging light source. The methods include detecting acoustic waves generated by light-absorbing components in the tissue area responsive to illumination by the pulse of light. The methods include generating, based on the detected acoustic waves, an image. The methods include providing, through a user interface, blood oxygenation information about the tissue area based on the image, which information is indicative of an effectiveness of the gas enrichment therapy or supersaturated oxygen therapy.

In certain implementations, the methods include controlling delivery of oxygen-enriched blood to the patient based on blood oxygenation information from the image

In certain implementations, the methods include tracking the image over time to determine a change in blood oxygenation in the tissue area over time.

In certain implementations, the methods include generating a tomographic image of the tissue area.

In certain implementations, the methods include generating a two-dimensional or three-dimensional image of the tissue area.

In a further example, methods for monitoring, analyzing, delivering and/or controlling gas enrichment therapy or supersaturated oxygen or gas therapy in a patient are disclosed. The methods include receiving, by a processor, one or more signals, the one or more signals corresponding to a measured value of one or more physiological parameters from at least one sensor. The methods may include generating, based on the measured value, an alert, optionally through a user interface, indicating a measured value of the physiological parameter indicative of an effectiveness of the gas enrichment therapy or supersaturated oxygen therapy. The methods may include controlling delivery of gas-enriched blood to the patient based on the one or more signals or the measured value. The methods may be performed as a computer implemented method. The methods may be performed using any of the implementations described previously with reference to the preceding example methods. In certain implementations, the physiological parameter is one or more of: a blood oxygen parameter, which may comprise arterial pO₂ and/or SO₂; arterial blood pressure and an electrical activity of the heart measured (which may be measured by an ECG sensor). The one or more signals may comprise: signals from an ECG sensor measuring electrical activity of the heart; a measured impedance value from a tissue area between a plurality of internal electrodes; a measured impedance value from a tissue area between a plurality of external electrodes; a measured impedance value from a tissue area between one or more internal electrodes and one or more external electrodes; one or more signals corresponding to a level of a target molecule in the target tissue; a measured fluorescence of a sensor molecule on an O₂ fluorescence probe; signals corresponding to a measured value of pO₂ in the tissue area from a transcutaneous pO₂ probe; signals corresponding to detected acoustic waves.

In a further example, methods for monitoring, analyzing, delivering and/or controlling gas enrichment therapy or supersaturated oxygen or gas therapy in a patient are disclosed. The methods include measuring, via one or more sensors, one or more physiological parameters of the patient. The methods include transmitting one or more signals to a processor, the one or more signals corresponding to a measured value of the one or more physiological parameters from the at least one sensor. The methods include generating, based on the measured value, an alert through a user interface indicating a measured value of the physiological parameter indicative of an effectiveness of the gas enrichment therapy or supersaturated oxygen therapy.

In relation to any of the above examples, there may be provided an implementation in which the gas enriched liquid comprises a supersaturated oxygen liquid. The supersaturated oxygen liquid may have has an O2 concentration of 0.1-6 ml O2/ml liquid (STP).

In relation to any of the above examples, there may be provided an implementation in which the gas-enriched blood comprises a supersaturated oxygen enriched blood. The supersaturated oxygen enriched blood may have a pO2 of 600-1500 mmHg.

According to a further example, there may be provided a computer program product, or non-transitory computer readable medium, comprising computer program instructions configured to cause a processor to perform the computer implemented methods described previously with respect to any of the example methods. It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1A shows one implementation of a system for delivering gas enrichment therapy or supersaturated oxygen therapy to a patient.

FIG. 1B shows a schematic diagram of the system of FIG. 1A.

FIG. 1C shows a schematic diagram of the system of FIG. 1A.

FIG. 2 shows a flow diagram of an example system for controlling delivery of oxygen enriched blood to a patient based on a sensor detecting one or more parameters in blood.

FIG. 3A shows a flow diagram of an example system for controlling delivery of oxygen enriched blood to a patient based on a tomographic map of measured impedance values.

FIG. 3B shows a schematic of a system with an impedance tomography catheter for use with the system of FIG. 3A.

FIG. 4A shows a flow diagram of an example system for controlling delivery of oxygen enriched blood to a patient based on measurements from a nuclear magnetic resonance probe.

FIG. 4B shows a schematic of a system with a nuclear magnetic resonance probe for use with the system of FIG. 4A.

FIG. 5A shows a flow diagram of an example system for controlling delivery of oxygen enriched blood to a patient based on measurements from an O₂ fluorescence probe.

FIG. 5B shows a schematic of a system with an O₂ fluorescence probe for use with the system of FIG. 5A.

FIG. 6A shows a flow diagram of an example system for controlling delivery of oxygen enriched liquid into a patient's blood vessel based on measurements from a transcutaneous pO₂ probe.

FIG. 6B shows a schematic of a system with a transcutaneous pO₂ probe for use with the system of FIG. 6A.

FIG. 7A shows a flow diagram of an example system for controlling delivery of oxygen enriched blood to a patient based on blood oxygenation information from a photoacoustic image.

FIG. 7B shows a schematic of a system with a photoacoustic imaging probe for use with the system of FIG. 7A.

The features and advantages of the inventive subject matter disclosed herein will become more apparent from the detailed description set forth below when taken in conjunction with the drawings.

### DETAILED DESCRIPTION

The following disclosure describes systems and methods related to, and example embodiments of, gas enrichment therapy or supersaturated oxygen or gas therapy systems, methods and components. The systems permit supersaturated oxygen (SO₂) therapy to be provided to patients and controlled based on an analysis of one or more patient parameters. SSO₂ therapy refers to minimally invasive procedures for enriching oxygen content of blood through catheter-facilitated infusion of oxygen-supersaturated physiological fluid (e.g., blood) or infusion of oxygen-supersaturated liquid, such as saline, directly into a patient's blood vessel. These procedures generally are aimed at treating a patient who has suffered an acute myocardial infarction (AMI), but can be used for other conditions, including peripheral vascular disease as well. There is a need for enhanced control of SSO₂ therapy based on feedback regarding patient blood oxygen parameters detected by sensor probes or catheters positioned on or in a patient. There is also a need to utilize imaging technologies to map the target tissue and provide feedback regarding changes in blood perfusion, tissue ischemia and infarct size in response to the SSO₂ therapy. The various feedback mechanisms described herein provide enhanced control of SSO₂ therapy and allow the caregiver and/or system to optimize SSO₂ therapy for improved patient outcomes.

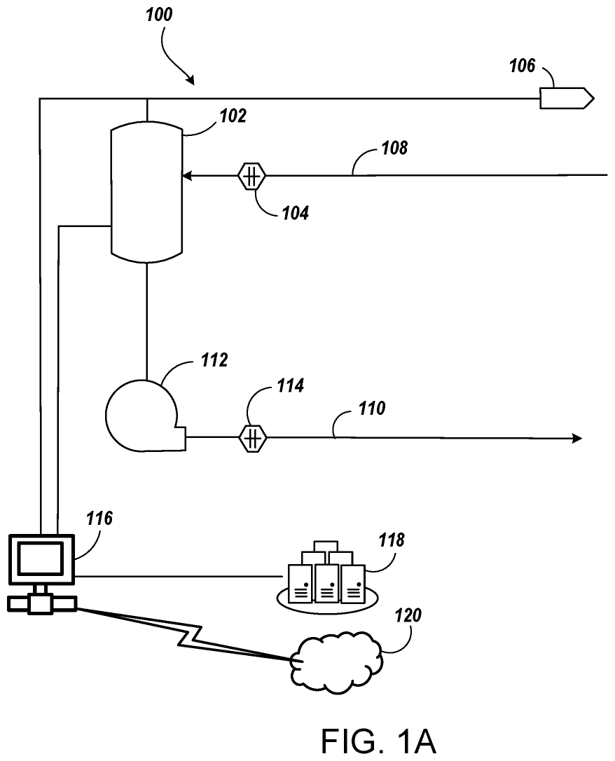

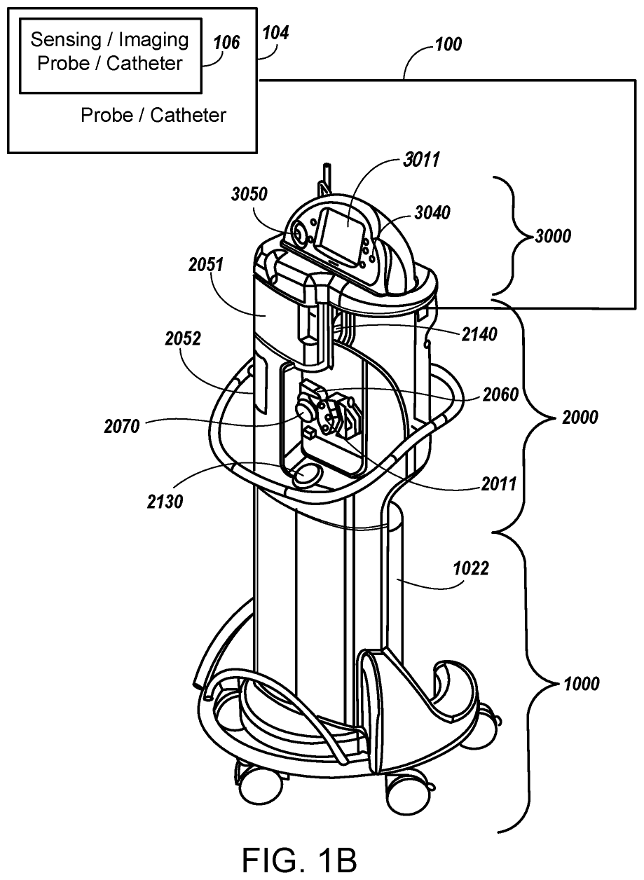

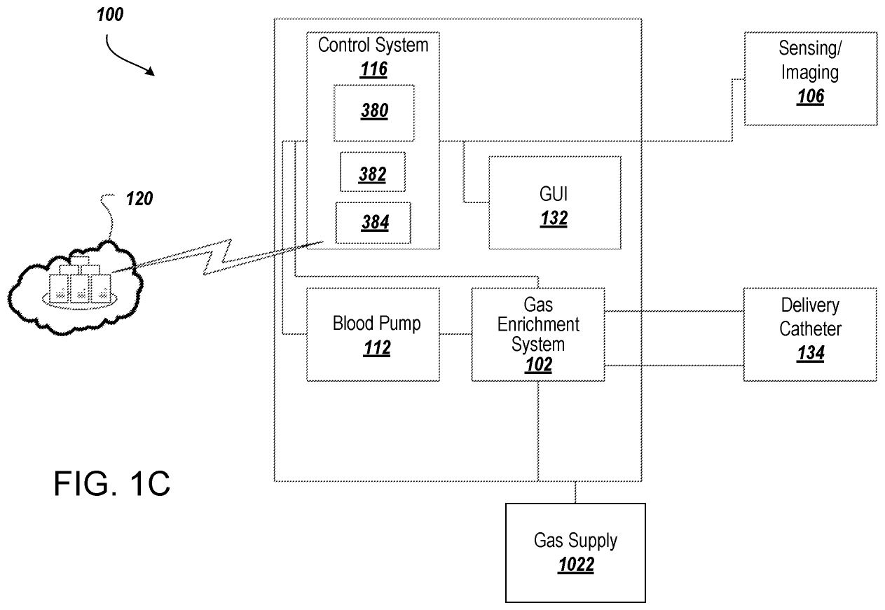

FIG. 1A illustrates a schematic of a system 100 for delivering gas enrichment therapy or supersaturated oxygen or gas therapy to a patient. The system 100 includes a gas enrichment system 102, described in further detail in FIG. 1B. The gas enrichment system 102 is used to infuse oxygen into the blood of a patient. The system 100 receives arterial blood from a patient via a conduit 102. One or more sensors 106 is coupled to the first arterial line 108 to detect a property of the blood being received from the patient. The sensor(s) 106 and/114 can measure various blood parameters, e.g., oxygen level, flow rate, pressure, hemoglobin content, hematocrit content, pH, CO₂ level, pO₂, SO₂, oxygen concentration, and/or temperature of blood arriving from the patient and entering the gas enrichment system 102. A blood pump 112, draws blood into the gas enrichment system 102, where the blood is mixed with a supersaturated oxygen liquid, e.g., saline, and pumps the resulting oxygen enriched blood or oxygen-supersaturated blood back to the patient via second arterial line 10.

As an example, the system 100 can be used to create a gas enriched blood by enriching a patient's blood with a gas enriched liquid, e.g., oxygen enriched liquid, in the gas enrichment system 102 to form gas enriched blood, e.g., oxygen enriched blood, and deliver the gas enriched blood to a patient, e.g., in the case of oxygen, delivering oxygen enriched blood to a patient, thereby increasing oxygen in the blood of the patient and diffusion of oxygen into tissue. In certain implementations, oxygen enriched liquid or solution, e.g., supersaturated oxygen liquid or solution (also referred to as oxygen supersaturated liquid or supersaturated oxygen fluid), may include liquid having a dissolved O₂ concentration of 0.1 ml O2/ml liquid (STP) or greater or 0.1-6 ml O2/ml liquid (STP) or 0.2-3 ml O2/ml liquid (STP) (e.g., without clinically significant gas emboli). When such supersaturated oxygen liquid or solution is mixed with blood, the resulting blood may be referred to as supersaturated oxygen enriched blood (also referred to as oxygen supersaturated blood). In certain implementations, the system 100 may deliver an infusion of supersaturated oxygen enriched blood having an elevated pO₂ in a target range of 400 mmHg or greater or 600-1500 mmHg or 760-1200 mmHg or around 1000 mmHg.

In one example, supersaturated oxygen enriched blood may have a pO2 of 760-1500 mmHg when a source blood delivered to the gas enrichment system for mixing with a supersaturated oxygen liquid has a minimum pO₂ of 80 mmHg, the blood flow rate is 50-150 ml/min, the SSO2 saline flow rate is 2-5 ml/min and the dissolved O₂ concentration in saline is 0.2-3 ml O2/ml saline (STP).

In another example, where the source blood is below 80 mmHg, the treatment objective may be to boost the blood pO2 to above 80 mmHg, so the system 100 may deliver an infusion of supersaturated oxygen enriched blood having a pO2 level of 80 mmHg or greater or 80-760 mmHg.

In addition to or as an alternative to the sensor 106 on the first arterial line, the second arterial line 110 may include one or more sensors 114 positioned therein for measuring various blood parameters or analyzing the enriched blood before it is pumped back to the patient. The system includes one or more control systems 116, that can receive and compare information obtained from the sensors 106 and 114, via wired or wireless connection to the control system, and can be used to control the blood pump 112 and the gas enrichment system 102. For example, the control system can receive a signal from the second arterial line sensor 114 corresponding to a value of the measured partial pressure of oxygen or pO₂ in blood flowing from the gas enrichment system to the patient. The control system compares the measured pO₂ to a target range of blood pO₂ e.g., 760-1240 mmHg or 760-1500 mmHg. For example, the 760-1240 mmHg or 760-1500 mmHg target range may be calculated based on a preprogrammed blood flow rate of 50-150 ml/min, saline flow rate of 2-5 ml/min and dissolved O₂ concentration in saline of 0.4-1.5 ml O₂/ml saline (STP) or 2-3 ml O₂/ml saline (STP). The control system can adjust any of the above parameters based on the measured pO₂ in blood to achieve an arterial blood pO₂ within the target range. The control system 116 can be communicably coupled to one or more local server systems, which can be configured for data storage locally and/or communicably coupled to one or more remote server systems 118 via a network such as the internet 120. The control system 116 can also include user interface components such as a display, keyboard, or mouse. These components can be used to adjust various parameters and view various reports that may be generated and/or displayed based on the processes executed by the control system.

The system 100 also includes a sensing probe 106. As discussed in further detail herein, the sensing probe can be used for measuring patient's physiological parameters, e.g., blood or tissue parameters, imaging, or optical sensing, and can be embodied in a catheter for sensing patient parameters internally or can be in the form of another probe or sensor system for sensing from inside or outside of the body, e.g. a transcutaneous pO₂ sensor probe. In certain implementations, the sensor system can be a combination of internal and external sensor components. The sensor may be coupled to the control system via a cable or other wired connection.

The sensing probe 106 provides information related to one or more physiological parameters. The information is analyzed by the control system 116, which controls and adjusts the infusion of the gas enrichment system 102 and the pumping of blood pump 112 based on the information obtained from the sensor.

FIG. 1B shows the system 100 of FIG. 1A for administering Supersaturated Oxygen (“SO₂”) therapy in greater detail. The system 100 for administering SSO₂ therapy generally includes three component devices: the main control system, the gas enrichment system (e.g., oxygenation cartridge), and the infusion device (e.g., an infusion catheter). These devices function together to create a highly oxygen-enriched saline solution called SSO₂ solution. A small amount of autologous blood is mixed with the SSO₂ solution producing oxygen-supersaturated blood. The oxygen-supersaturated blood is delivered to the patient. The system 100 may have a modular design comprising three removable modules, the base module 1000, the mid-section control module 2000, and the display module 3000. The system 100 also has a sensing and/or imaging probe 106, which can be implemented via a catheter in accordance with certain implementations. A gas tank receptacle is provided on the backside of the base module 1000 for receiving and housing a standard “E-bottle” USP oxygen tank 1022. The oxygen tank 1022 is mounted to the system via a gas tank adapter. A suitable gas, such as oxygen, is delivered from the oxygen tank 1022, to a second chamber within an oxygenation cartridge. The physiologic liquid, e.g., saline, from a first chamber is pumped into the second chamber and atomized to create an oxygen-supersaturated physiologic solution. This oxygen-supersaturated physiologic solution is then delivered into a third chamber of the oxygenation cartridge along with the blood from the patient. As the patient's blood mixes with the oxygen-supersaturated physiologic solution, oxygen-supersaturated or enriched blood is created and then delivered to a targeted major epicardial artery, e.g., the left main coronary artery, via an infusion catheter.

The system 100 includes a fluid pump assembly including a pump 112. The pump assembly may also include a drawtube, a pressure sensor, a bubble detector/flow meter (2060), a return clamp (2070), and a return tube. A cartridge housing is configured to receive a matching cartridge (i.e. the gas-enrichment system). The cartridge housing includes various sensing, controlling, and interfacing mechanisms for use with the cartridge. In certain implementations, the gas-enrichment system is configured as a direct injection system rather than a cartridge.

Each of the three modules (1000, 2000, and 3000) of the system 100 may include doors or access panels for protecting and accessing the various components housed therein. For example, the mid-section control module 200 includes a hinged door 2051 for enclosing the gas-enrichment system (i.e. the cartridge) and access panel 2052 for covering the access window to the internal space of the module. A safety switch (e.g. an emergency stop switch 3050) may be provided so that a user can initiate a shutdown of the system in the same fashion even if the system is operating within its prescribed bounds.

In the above particular embodiment, the body of the base module 1000 is made up of a tubular chassis situated on a circular-shaped pedestal 1001. A plurality of wheels 1002 are mounted on the bottom of the circular-shaped pedestal to provide mobility for the system. The wheels have a locking mechanism for keeping the wheels stationary. The base chassis houses certain electrical and mechanical components including a battery 1003 (not shown), a power supply 1004 (not shown), and connectors for connecting the base module 1000 to the mid-section main module 2000.

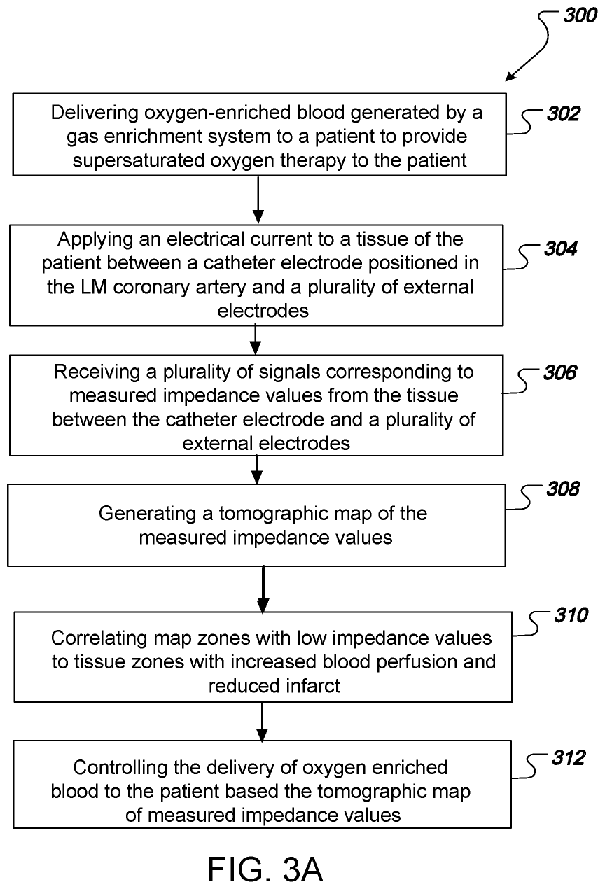

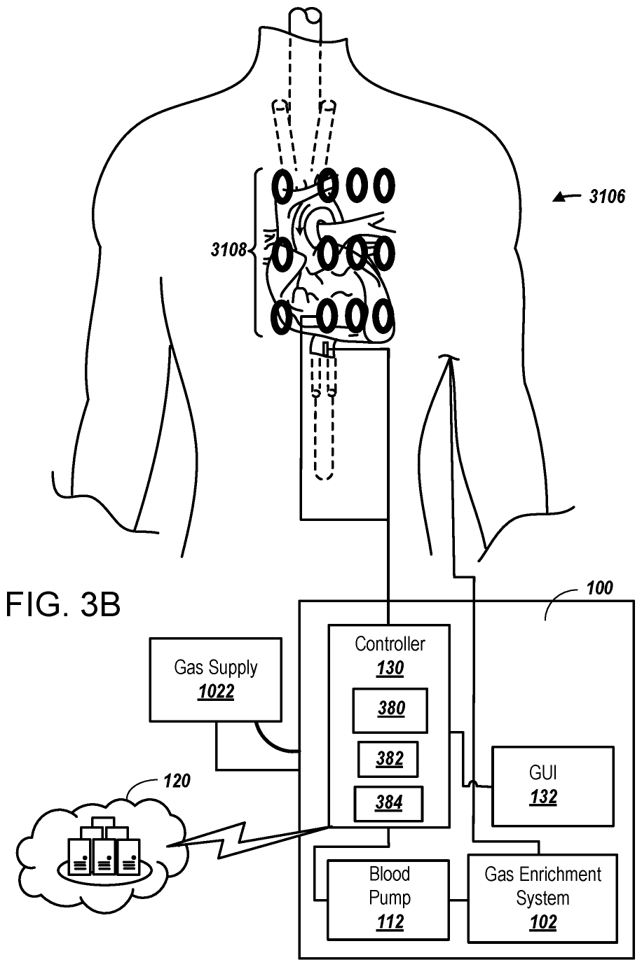

FIG. 1C shows the system 100 schematically. As demonstrated in FIG. 1C, the system 100 includes the gas enrichment system 102 that can be implemented in various forms, such as the three chambered cartridge described above. The gas enrichment system is supplied with gas via gas supply 1022, which can be in the form of an onsite storage tank as illustrated in FIG. 1B. The system 100 also includes a sensing/imaging component or system 106, which may be coupled to the controller via a cable or other wired connection, which may also be implemented in various forms as described in greater detail in connection with FIGS. 3B, 4B, 5B, 6B, and 7B. The sensing component 106 can measure physiological parameters, for example, one or more blood or tissue oxygen parameters of the blood of a patient. The system infuses the SSO₂ solution produced into blood, and delivers the oxygen-supersaturated blood to the targeted major epicardial artery via delivery catheter 134 as pumped by the blood pump 112. The infusion of the blood and the pumping of the blood are controlled by the controller 130, which includes a processor 380, a memory 382, and associated input and output circuitry 384 for communicably connecting with the sensing/imaging system 106, the gas enrichment system 102, blood pump 112 and the graphical user interface (GUI) 132. The controller 130 can receive input from the sensing system 106 and the gas enrichment system 102 and controls the gas enrichment system 102 responsive to inputs received from the sensing system as determined by one or more algorithms stored in the memory 382 of the controller 130 and processed by the processor 380 (e.g., or processor system). The processor 380 is configured to receive one or more signals corresponding to a measured value of one or more physiological parameters, e.g., blood oxygen parameters of the blood, from the sensing system 106 and generate, based on the measured value, an alert indicative of the physiological parameter or a characteristic of the physiological parameter, e.g., a level of the measured blood oxygen parameter. The alert can be indicated on the graphical user interface. The measured physiological parameter, e.g., the level of the measured blood oxygen parameter, is used by the processor 380 to control the supersaturated oxygen or gas therapy implemented by the system 100. The controller 130 may be communicably coupled to a network, such as the internet 120, through which various remote servers can be accessed for data storage and or information access. The communication network can be used to remotely control or monitor the system 100. A graphical user interface 132 is provided in the system 100 for interaction with the system by a user for control and monitoring of the various system components. The graphical user interface 132 can also be viewed or accessed via the network 120, e.g., the graphical user interface may provide remote alerts or prompts to a user.

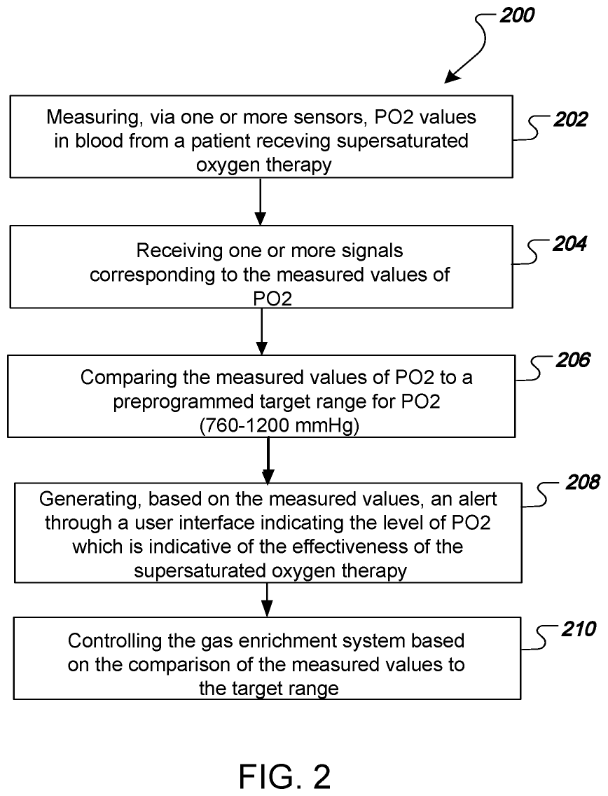

FIG. 2 shows a flow diagram of an example process 200 for controlling delivery of oxygen-enriched blood to a patient based on a sensor detecting one or more parameters in blood, which may be performed by one example of the system of FIG. 1C. In particular, the process 200 measures a blood oxygen parameter to control supersaturated gas therapy e.g., the delivery of the oxygen-enriched blood, to a patient. Sensing 106 may be performed by an oxygenation sensor that can be positioned in or on the conduit configured for blood flow from the gas enrichment device to the patient or in or on the conduit configured for blood flow from the patient to the gas enrichment device. The oxygenation sensor may be coupled to the controller via a cable or other wired connection. The oxygenation sensor may be non-invasive and utilize optical methods such as near infrared spectroscopy (NIRS) or a pulse oximeter for estimating pO₂ and/or oxygen saturation (SO₂), e.g., arterial pO₂ or SO₂ in the blood. Pulse oximetry estimates the percentage of oxygen bound to hemoglobin in the blood. A pulse oximeter uses light-emitting diodes and a light-sensitive sensor to measure the absorption of red and infrared light. All or a portion of the process 200 can be implemented via the controller 130 of FIG. 1C, i.e., using the processor 380 and memory storage device 382 to execute various actions. For example, at 202, the oxygenation sensor may be used to measure pO₂ values in blood from a patient receiving supersaturated oxygen or gas therapy. In certain implementations, the sensor comprises an electrode such as a Clark electrode for measuring pO₂. A Clark electrode is an electrode that measures ambient oxygen concentration in a liquid using a catalytic platinum surface according to the net reaction O₂+4e⁻+4H⁺→2H₂O. At 204, the processor 380 receives the signals from the oxygenation sensor that correspond to the measured values of pO₂, at 204. At 206, the processor 380 compares the measured pO₂ to a target range of blood pO₂, e.g., 760-1200 mmHg or 760-1500 mmHg. As stated supra, a 760-1240 mmHg or 760-1500 mmHg target range may be calculated based on a preprogrammed blood flow rate of 50-150 ml/min, saline flow rate of 2-5 ml/min and dissolved O₂ concentration in saline of 0.4-1.5 ml O₂/ml saline (STP) or 2-3 ml O₂/ml saline (STP). The control system can adjust any of the above parameters based on the measured pO₂ in blood to achieve an arterial blood pO₂ within the target range. At 208, the processor 380 generates an alert, e.g., through a user interface, that indicates the pO₂. The measured pO₂ indicates the effectiveness of the supersaturated oxygen or gas therapy, letting the caregiver know if the pO₂ in blood is within the preprogrammed target range for optimizing the delivery of oxygen to the patient's ischemic tissue. At 210, the processor 380 controls the gas enrichment system by modifying one or more of the above referenced saline or blood parameters to base on the sensor values.

In certain implementations, a system according to FIG. 1C may perform a process for controlling the delivery of oxygen-enriched blood to a patient based on feedback from a sensor configured to detect a patient's blood pressure (arterial or venous). The process measures a blood pressure to control supersaturated gas therapy e.g., the delivery of the oxygen-enriched blood, to a patient. Sensing may be performed by a pressure sensor that can be positioned in or on the conduit configured for blood flow from the gas enrichment device to the patient or in or on the conduit configured for blood flow from the patient to the gas enrichment device. The blood pressure sensor or blood pressure transducer may be coupled to the controller via a cable or other wired connection. All or a portion of the process can be implemented via the controller 130, i.e., using the processor 380 and memory storage device 382 to execute various actions. For example, the blood pressure sensor may be used to measure blood pressure values in blood from a patient receiving supersaturated oxygen or gas therapy. The processor 380 receives signals from the blood pressure sensor, which correspond to the measured values of blood pressure. The processor 380 compares the measured blood pressure to a target range of blood pressure, e.g., blood pressure in a healthy individual. The processor 380 generates an alert, e.g., through a user interface, that indicates the blood pressure. The measured blood pressure indicates the effectiveness of the supersaturated oxygen or gas therapy, letting the caregiver know if the blood pressure is within a target range in order to optimize the SSO₂ therapy. The processor 380 may control the gas enrichment system by modifying one or more saline or blood parameters in the gas-enrichment system to optimize therapy based on the blood pressure feedback.

Changes in blood pressure (e.g., arterial or venous) may provide feedback regarding the effectiveness of the SSO₂ therapy. For example, a change in blood pressure may be indicative of change in blood flow in myocardial tissue in response to the SSO₂ therapy. The SSO₂ therapy provides a high concentration gradient of O₂ that enables increased diffusive transfer to ischemic areas of myocardium. This diffusive transfer of O₂ to areas most in need does not depend on blood flow and thus SSO₂ can easily access the endothelial cells of capillaries suffering from edema (swelling). SSO₂ is able to reverse this edema response in the microvasculature and restore flow, nurturing surrounding heart tissue with oxygenated blood.

An example sensor for measuring an arterial pressure of the patient's blood includes a pressure sensor positioned in or coupled to the catheter. The catheter may be connected to a fluid-filled system or pressure tube, which is connected to an electronic pressure transducer and/or pressure monitor. A change in detected blood pressure may be indicative of improved perfusion and/or restored flow in ischemic tissue as a result of the SSO₂ therapy. The therapy may result in improved heart function. In certain implementations, the processor may control the delivery of supersaturated oxygen therapy based on the arterial pressure feedback.

In certain implementations, feedback may be based on a measured blood pressure waveform. A change in a waveform reflection pattern may be detected. In one example, changes in the reflection pattern of the normal pulsatile waveform of the patient's blood pressure may be detected or measured. In another example, a pulsatile flow may be created (for more fine tuning), and changes in the reflection patter of the created pulsatile waveform of the patient's blood pressure may be detected or measured. In either example, the pulsatile waveform may be analyzed for information, such as the relative magnitude and the timing of the secondary peak identified in that waveform.

In certain implementations, a system according to FIG. 1C may perform a process for controlling the delivery of oxygen-enriched blood to a patient based on feedback from one or more ECG sensors or electrodes. The process measures the electrical activity of a patient's heart using an ECG signal to control supersaturated gas therapy e.g., the delivery of the oxygen-enriched blood, to a patient. An ECG sensor or electrode may be positioned on a patient's chest. The ECG sensor may be coupled to the controller via a cable or other wired connection. All or a portion of the process can be implemented via the controller 130, i.e., using the processor 380 and memory storage device 382 to execute various actions. For example, the ECG sensor may be used to measure the electrical activity of a patient's heart, where the patient is receiving supersaturated oxygen or gas therapy. The processor 380 receives signals from the ECG sensor. The processor 380 compares the ECG signal to a target signal, e.g., the signal of a healthy individual. The processor 380 generates an alert, e.g., through a user interface, that indicates whether the ECG signal is normal or abnormal. The measured ECG signal indicates the effectiveness of the supersaturated oxygen or gas therapy, letting the caregiver know if the patient's ECG is normal or abnormal in order to optimize the SSO₂ therapy. For example, alleviation of ischemia for an acute cardiac patient can reverse abnormal ECG signals. The processor 380 may control the gas enrichment system by modifying one or more saline or blood parameters in the gas-enrichment system to optimize therapy based on the ECG feedback.

FIG. 3A shows a flow diagram of an example system 300 for controlling delivery of oxygen enriched blood to a patient based on a tomographic map of measured impedance values generated by an impedance tomography catheter sensor system 3106 shown in FIG. 3B. Electrical impedance tomography (EIT) is a noninvasive type of medical imaging in which the electrical conductivity, permittivity, and impedance of a part of the body is inferred from surface electrode measurements and used to form a tomographic image of that tissue region. At 302, system 300 causes delivery of gas-enriched blood generated by a gas enrichment system to a patient to provide supersaturated gas e.g., oxygen, therapy to the patient. At 304, an electrical current is applied to a tissue of the patient between a catheter electrode positioned in the left main (LM) coronary artery and a plurality of external electrodes positioned on the external surface of the patient's body. The catheter electrodes and external electrodes may be coupled to the controller via one or more cables or other wired connections. At 306, a processor 380 of the system 300 receives a plurality of signals from the electrodes that correspond to measured impedance values from the tissue between the catheter electrode and the plurality of external electrodes. At 308, the processor 380 generates a tomographic map of the measured impedance values. In one example, processor 380 may be used to generate a map of measured impedance values in a tissue area having an infarct and to compare the tomographic map of measured impedance values in that tissue area to a baseline tomographic map of measured impedance values in the tissue area to determine changes in blood perfusion or changes in infarct size in the patient throughout SSO2 therapy. The mapped areas and any changes in blood perfusion or changes in infarct size may be stored by the processor 380 in a memory device 382 and may be tagged for future reference. At 310, the processor 380 correlates mapped zones with low impedance values to tissue zones with increased blood perfusion and reduced infarct size. For ischemic tissue, a higher impedance would be expected because there would be less blood present in the ischemic tissue compared to non-ischemic tissue or tissue with increased blood perfusion. The processor 380 may also overlay and spatially align these mapped zones on other mapped images of the same area or the same infarct zone, such as MRI or CT images. At 312, the delivery of oxygen-enriched blood to the patient is controlled based on the tomographic map of measured impedance values. For example, the location of the delivery catheter (which in certain implementations can be combined with the catheter housing the catheter electrode) can be adjusted and/or the rate of delivery of the oxygen-enriched blood (i.e. as controlled by a blood pump of the system) can be adjusted. In certain embodiments, if the tomographic map has regions not showing a decrease in impedance (therefore, an increase in perfusion) of less than a predetermined threshold, e.g. 10%, or if the estimated infarct size based on the tomographic map has not been reduced by more than some other predetermined threshold, e.g. 15%, the SO₂ in the blood can be increased based on the tomographic map of measured impedance values and/or the deduced changes in infarct size. The SO₂ in the blood can be decreased or stopped based on exceeding a threshold in either the impedance of a region of the tomographic map or the estimated infarct size.

FIG. 3B shows a schematic of the system 100 employing an impedance tomography catheter as described in connection with FIG. 3A. In FIG. 3B a sensing catheter 3106 is inserted into a lumen of the patient. In particular, the sensing catheter 3106 is positioned in the LM coronary artery of the patient; however, in other embodiments the sensing catheter may be positioned in other coronary arteries, or alternatively in other lumens, such as the esophagus. The sensing catheter includes one or more electrodes disposed along a length of the catheter, e.g., a pair of electrodes may be utilized. A plurality of additional surface electrodes (e.g., electrodes 3108) are disposed externally on the thorax of the subject. The plurality of electrodes are disposed at spaced apart positions. The use of multiple electrodes disposed in spaced apart positions along the length of the catheter 3106 permits impedance measurements of tissue between the catheter electrodes and the surface electrodes to be made in multiple axes.

In another embodiment, the sensing catheter may include a plurality of electrodes. The electrodes may be any type of electrode suitable for use inside the body of a subject, and may be mounted to an external surface of the catheter, or integrated therein. Rather than including pairs of electrodes disposed on opposing side surfaces of the probe, a single electrode may be provided at each level along the length of the catheter, and that in certain embodiments, only a single electrode may be provided. However, the use of multiple electrodes disposed in spaced apart positions along the length of the probe permits impedance measurements to be made in multiple axes as stated supra.

The plurality of additional electrodes may be any type of electrode suitable for external use on the body of the subject, such as wet or dry self-adhesive medical electrodes typically used to measure electrical signals on the body of a subject. The Sheffield Mark 3.5 and the Enlight 1800 are exemplary electric impedance tomography technologies that may be implemented to provide imaging feedback useful for controlling the SSO₂ therapy delivered by the system 300.

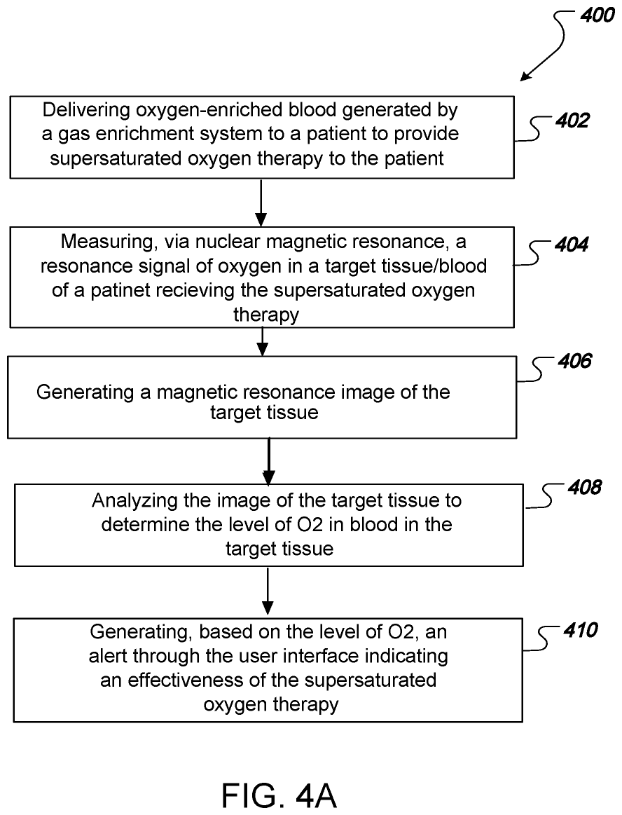

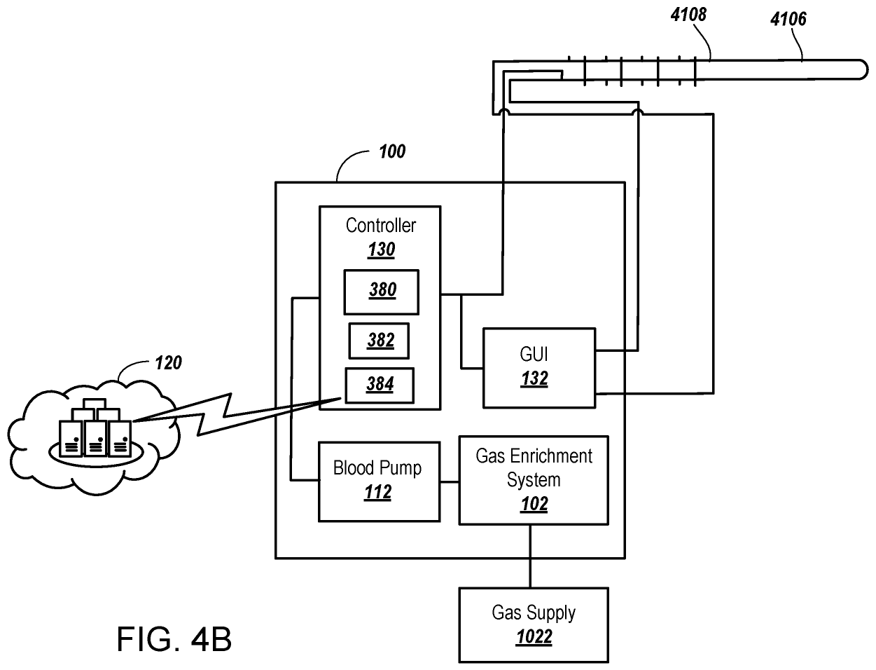

FIG. 4A shows a flow diagram of an example system 400 for controlling delivery of oxygen enriched blood to a patient based on measurements from a nuclear magnetic resonance (NMR) probe 4106 shown in FIG. 4B. At 402, system 400 causes delivery of gas-enriched blood generated by a gas enrichment system to a patient to provide supersaturated oxygen or gas therapy to the patient. At 404, a nuclear magnetic resonance probe (e.g. probe 4106 shown in FIG. 4B), which may be coupled to the controller via a cable or other wired connection, is used to measure one or more tissue parameters in a target tissue of a patient receiving the supersaturated oxygen or gas therapy, e.g., to measure a resonance signal of a target molecule in tissue, such as blood oxygen. At 406, a processor (e.g., processor 380) of the system generates a magnetic resonance image (MRI) of the target tissue based on the detected resonance signals of the target molecule detected by the nuclear magnetic resonance probe and transmitted to the processor 380 from the nuclear magnetic resonance probe. At 408, the processor 380 is used to analyze the image of the target tissue to determine the level of the target molecule or changes in the level of the target molecule, such as the level of or changes in SO₂ in blood in the target tissue. The target tissue may be an infarct zone of the heart. The analysis is used at 410 by the processor 380 to generate an alert through a user interface. The alert provides information indicating an effectiveness of the supersaturated oxygen or gas therapy based on the presences or level of the target molecule in the target tissue. Based on the alert, the delivery of oxygen-enriched blood to the patient can be controlled. For example, the location of the delivery catheter can be adjusted and/or the rate of delivery of the gas-enriched blood (i.e. as controlled by a blood pump of the system) can be adjusted. Alternatively, the MRI image may be displayed on the user interface or a remote monitor for the caregiver to analyze, and the caregiver may control therapy based on the image.

As stated above, the target molecule may be oxygen. In another embodiment, the target molecule may be a high-energy phosphate. Examples of high-energy phosphate molecules include adenosine triphosphate (ATP), adenosine di-phosphate (ADP) adenosine monophosphate (AMP) and free phosphate ion (Pi). Monitoring the amount of these molecules in the target tissue, e.g., tissue that has been affected by an infarct, would provide an indication of how abnormal the energetic/metabolic state of the target tissue is, which is indicative of the presence of or changes in tissue ischemia. The more ischemic, the lower the energy/metabolic state of the tissue. The supersaturated oxygen or gas therapy may be controlled based on the detected energetic/metabolic state of the target tissue and the detected metabolic recovery of the tissue.

In one embodiment, a magnetic resonance receiver, e.g., a coil, may be positioned on the end of a catheter and the magnetic resonance driving signal would be generated from elsewhere in the system, e.g., in a position external to the patient. This embodiment would allow for focused detection the target tissue of interest.

In another embodiment a magnetic resonance receiver, e.g., a coil, and the magnetic resonance driver can be located on the catheter. The magnetic resonance catheter may be the same catheter positioned in the femoral artery or left main or other coronary artery, which is used to deliver the gas-enriched blood. Alternatively, the magnetic resonance catheter may be a separate catheter, e.g., positioned in a ventricle or other vessel.

FIG. 4B shows a schematic of a system with a nuclear magnetic resonance probe for use with the system of FIG. 4A. In particular, the system 100 can be implemented with the magnetic resonance imaging (MRI) catheter 4106 as the sensing element. The catheter 4102 includes a magnetic coil 4108 wound around a peripheral portion of the catheter 4106. The magnetic coil 4108 is connected to the controller 130 for activation and for receipt of imaging data therefrom. The imaging data obtained from the magnetic coil 4108 is processed by the processor 380 of controller 130 and can be displayed on the GUI 132. The processor 380 receives signals corresponding to a level of a target molecule in the target tissue based on the measured resonance signal of the molecule from the nuclear magnetic resonance imaging probe. Although the catheter 4106 is an MRI catheter, it can still be implemented as the delivery catheter for delivering the SSO₂ blood to the patient. In another embodiment, the magnetic coil may be integrated in the catheter. In another embodiment, the magnetic resonance probe may be separate from the catheter, e.g., a separate probe.

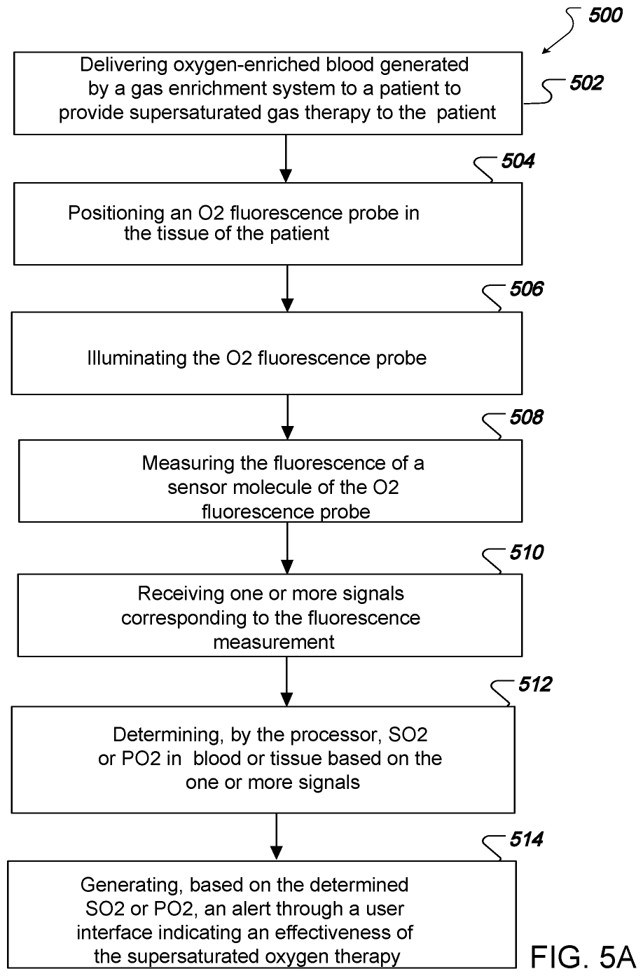

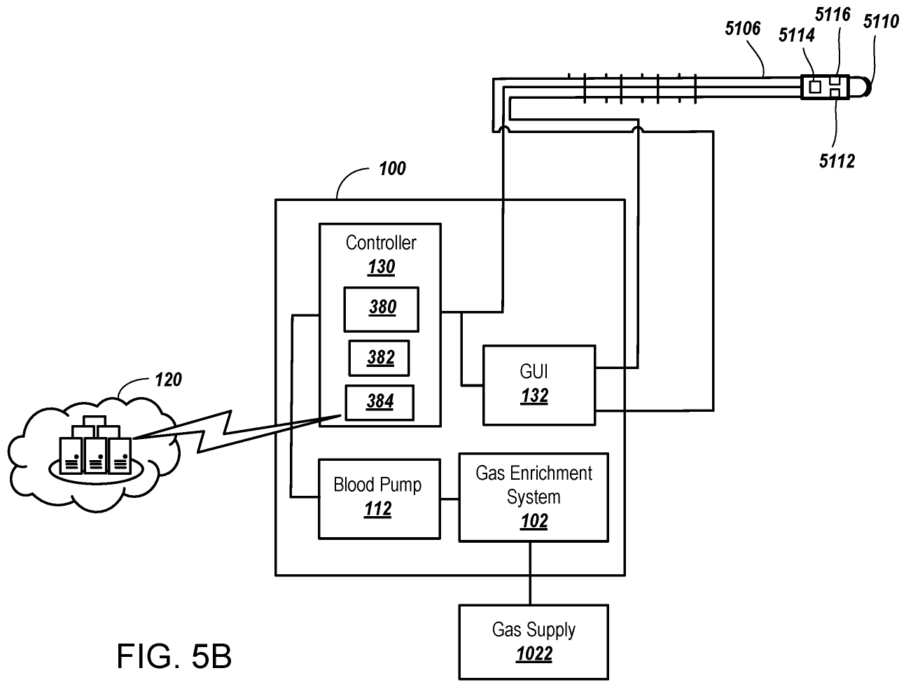

FIG. 5A shows a flow diagram of an example system 500 for controlling delivery of oxygen enriched blood to a patient based on measurements from a fluorescence probe as a sensing element as shown in FIG. 5B. The fluorescence probe may be coupled to a controller of the system via a cable or other wired connection. The fluorescence probe may be an O₂ fluorescence probe. At 502, system 500 causes delivery of gas-enriched blood generated by a gas enrichment system to a patient to provide supersaturated oxygen or gas therapy to the patient. The O2 fluorescence probe may be positioned in the conduit for blood flow to the gas-enrichment device, the conduit for blood flow from the gas-enrichment device to the patient, in a vessel in a target tissue of the patient, e.g., coupled to the gas-enriched blood delivery catheter, or inserted in or near target tissue at 504. At 506, a light source of the O₂ fluorescence probe is illuminated. A fiber optic cable can be used to provide light to the light source in certain implementations, where the fiber optic cable is connected to the controller of the system. At 508, the fluorescence of a sensor molecule of the O₂ fluorescence probe is measured. The sensor molecule can include fluorophore. A signal is received by the processor 380 of controller 130 from the O₂ fluorescence probe based on the fluorescence measurement at 510. Fluorescence is measured by measuring the lifetime or decay of the fluorescence intensity signal from the illuminated sensor molecule (e.g., fluorophore) on the fluorescence probe. The decay of this signal is caused by the quenching effect of oxygen molecules in the blood or in tissue on the fluorescence intensity signal of the sensor molecule. At 512, the processor 380 can determine the SO₂ or pO₂ in blood or tissue based on the quenching effect of oxygen on the florescence intensity signal of the florescence probe. Changes in the amount of time that is required for the signal to decay due to oxygen quenching are indicative of the local SO₂ or pO₂ in blood or tissue. At 514, the processor 380 generates an alert based on the determined SO₂ or pO₂ in blood or tissue. The alert indicates effectiveness of the supersaturated oxygen and can be provided via the user interface 132.

In certain embodiments, oxygen can quench the fluorescence or phosphorescence of a fluorophore or phosphor signal on a sensing probe. For example, a sensor molecule (fluorophore or phosphor) may be coated onto the end of a fiber optic bundle, optionally, one or more fibers. The sensor molecule is excited by a light source, which may be pulsed at a high frequency. The fluorescence or phosphorescence lifetime or decay is measured. Changes in the amount of time that is required for the signal to decay are indicative of the local oxygen concentration, SO₂ or pO₂ in blood or tissue. One of the advantages of using an O₂ fluorescence or phosphorescence probe for feedback is that it is possible to surround the sensor molecules with molecules that protect the sensing molecule from oxygen. In this way, the fluorescence or phosphorescence lifetime can be tuned for a wide range of oxygen concentrations, SO₂ or pO₂ values, particularly very high oxygen concentrations. The resulting SO₂ or pO₂ can be determined for the blood or tissue downstream of the oxygen enriched blood delivery site.

FIG. 5B shows a schematic of a system with an O₂ fluorescence probe for providing oxygen concentration, SO₂ or pO₂ feedback during supersaturated oxygen therapy. The O₂ fluorescence probe is for use with the system of FIG. 5A. The probe 5116 is coupled to the system controller via a wired connection. The probe includes a luminescence coating 5110, e.g., a coating comprising fluorophore molecules, a light sensor 5112, a first reference light source 5114 (e.g. a light emitting diode) and optionally a second reference light source 5116. The light sensor 5112 measures light reflected from the luminescence coating 5110, which light is projected by the first reference light source 5114. The reflected light from the luminescence coating is quenched by the oxygen in the blood or tissue with which the probe is in contact. Changes in the amount of time that is required for the signal to decay as a result of the oxygen quenching are indicative of the local oxygen concentration, SO₂ or pO₂ values in the blood or target tissue, e.g., myocardial tissue, in which the probe is inserted. In another embodiment, the O₂ fluorescence probe may be separate from the catheter, e.g., a separate probe such as the NEOFOX-GT or Unisense MicroOptode technology may be utilized.

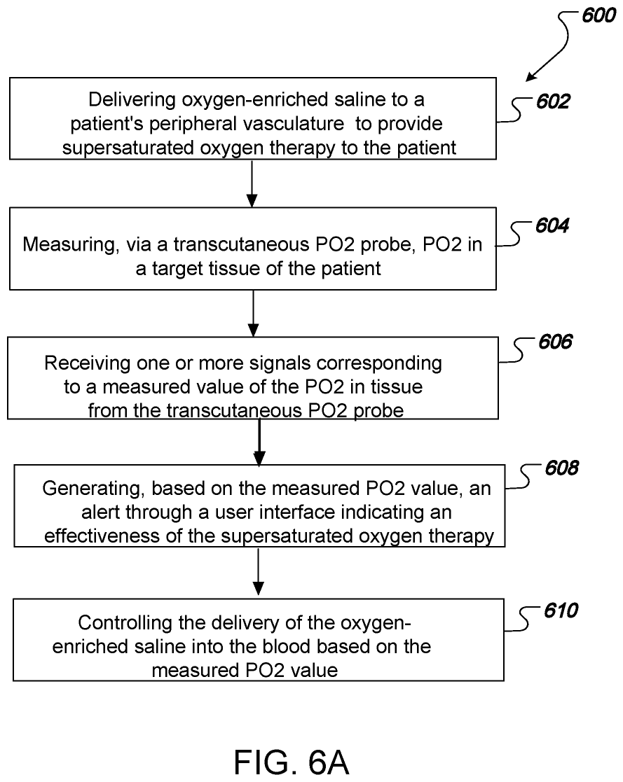

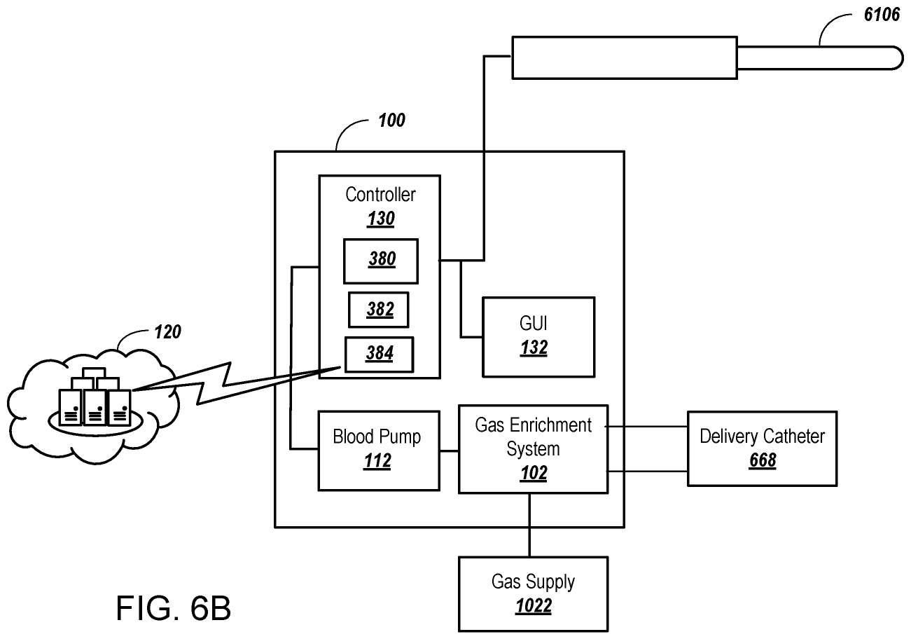

FIG. 6A shows a flow diagram of an example system for controlling delivery of oxygen enriched liquid into a patient's blood vessel based on measurements from a transcutaneous pO₂ probe. At 602, the system 100 causes delivery of oxygen-enriched saline to be delivered to a patient's peripheral vasculature to provide supersaturated oxygen therapy to the patient. At 604, a transcutaneous pO₂ probe (e.g., probe 6106 of FIG. 6B) may be inserted into a target tissue near the site of oxygen-enriched saline delivery. The transcutaneous pO₂ probe is used to measure pO₂ in the target tissue of the patient. At 606, the processor 380 of the controller 130 receives one or more signals corresponding to the measured value of the pO₂ in the tissue as determined by the transcutaneous pO₂ probe. The processor 380 generates, at 608, an alert based on the measured pO₂ value. The alert can be provided via a user interface to show the effectiveness of the supersaturated oxygen therapy based on the pO₂ in the target tissue. The processor 380 can also be used to control the delivery of the oxygen-enriched saline into the blood based on the measured pO₂ values, e.g., the processor 380 may control the flow rate of oxygen enriched saline and/or the concentration of O2 in saline.

FIG. 6B shows a schematic of a system with a transcutaneous pO₂ probe for use with the system of FIG. 6A. The system may be similar to the direct injection system described in U.S. Pat. No. 9,919,276, which delivers a supersaturated oxygen solution directly to a patient blood vessel, where the supersaturated oxygen mixes with the blood to form oxygen-enriched blood inside the patient's vasculature. The system of FIG. 6B includes a controller 130, having a processor 380, memory 382, and input/output circuity 384. The processor 380 receives signals from the pO₂ probe. The system includes a pump 612 for pumping liquid, such as saline into the gas enrichment system 602, in which saline is infused with oxygen gas to create the supersaturated oxygen solution SSO₂. The SSO₂ is then delivered via delivery catheter 668 directly to the patient's vasculature where it mixes with the patient's blood to create oxygen-enriched blood. The transcutaneous pO₂ probe 61606 is coupled to the controller by a wired connection. The transcutaneous pO₂ probe allows for non-invasive measurement of pO₂. The system includes a user interface 132 for displaying an alert indicating an effectiveness of the supersaturated oxygen therapy based on the measured pO₂ feedback. Accuracy of the pO₂ probe 6106 can be increased with constant local vasodilation by heating the skin at the site of application of the pO₂ probe 6106. This causes maximal blood flow in the skin. In some implementations, the transcutaneous pO₂ probe 6106 includes a combined platinum and silver electrode covered by an oxygen-permeable hydrophobic membrane, with a reservoir of phosphate buffer and potassium chloride trapped inside the electrode. A small heating element may be located inside the silver anode. The transcutaneous pO₂ probe 6106 can be applied to the anterior chest wall or other acceptable site and heated for measurements. In other embodiments, the transcutaneous probe may be a fluorescence probe.

In another embodiment, a skin contact probe for measuring gross pO₂ in tissue may be utilized to provide feedback regarding the SSO₂ therapy. The probe may be applied directly to the skin and provides a gross measurement of tissue oxygenation close to the skin. This measurement could be useful for the application of SSO₂ to treat peripheral vascular disease. Gross pO₂ of tissue in healthy individuals is around 40 mmHg. The processor may compare the measured gross pO₂ in a target tissue to the pO₂ in a healthy individual and adjust the delivery of SSO₂ therapy accordingly.

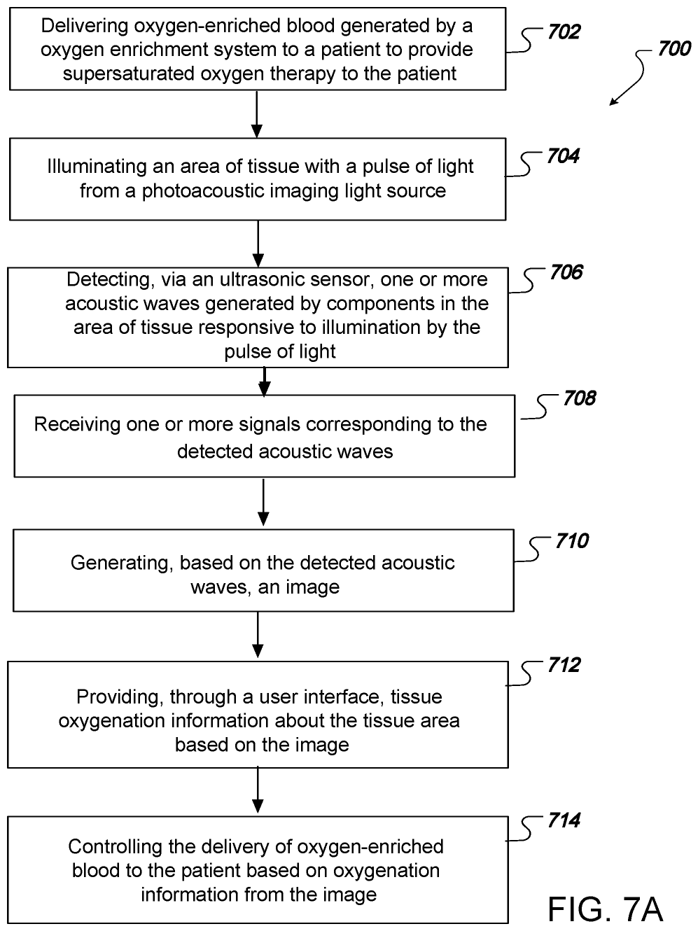

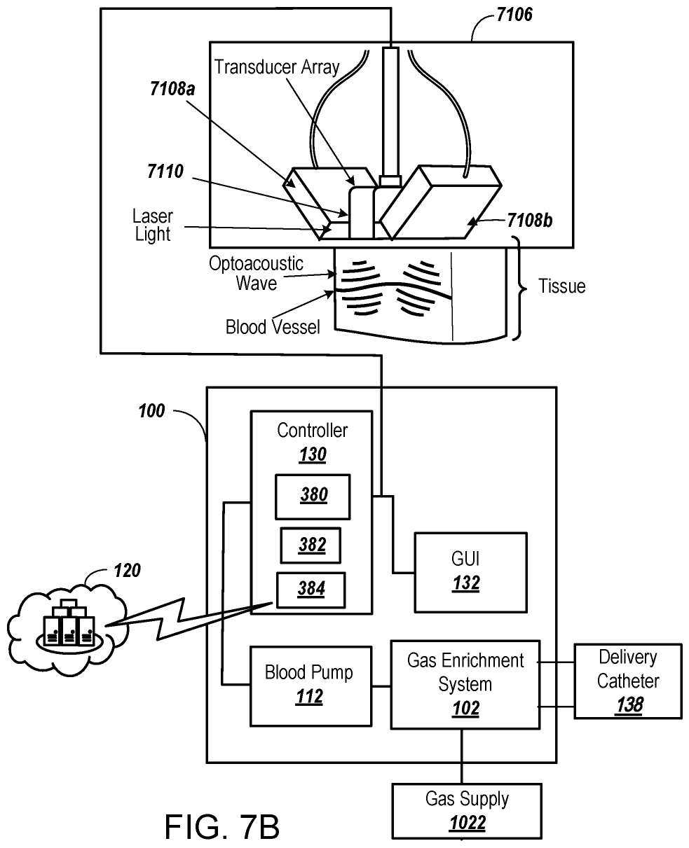

FIG. 7A shows a flow diagram of an example system for controlling delivery of oxygen enriched blood to a patient based on tissue oxygenation information from a photoacoustic image. At 702, system 700 causes delivery of oxygen-enriched blood generated by an oxygen enrichment system to a patient to provide supersaturated oxygen therapy to the patient. At 704, the system 700 causes a photoacoustic probe (e.g., a non-invasive photoacoustic probe 7106 shown in FIG. 7B) to illuminate an area of tissue with a pulse of light from a photoacoustic imaging light source. At 706, an ultrasonic sensor of the photoacoustic probe or a separate ultrasonic sensor detects one or more acoustic waves generated by molecules or structures in the area of tissue in response to illumination by the pulse of light. At 708, the processor 380 of the controller receives one or more signals corresponding to the detected acoustic waves from the ultrasonic sensor, which is coupled to the controller by a wired connection, and generates at 710, based on the detected acoustic waves, an image. At 712, the system 700 provides tissue oxygenation information about the tissue area based on the image through a user interface. The system 700 may use the tissue oxygenation information at 714 to control the delivery of oxygen-enriched blood to the patient.

FIG. 7B shows a schematic of a system 100 used with a photoacoustic imaging probe 7106 for detecting properties of tissue. Photoacoustic imaging is a medical imaging modality that uses optical excitation and acoustic detection to generate images of tissue structures based upon optical absorption within a tissue sample. A photoacoustic image can be regarded as an ultrasound image in which the contrast depends not on the mechanical and elastic properties of the tissue, but its optical properties, specifically optical absorption. It offers greater specificity than conventional ultrasound imaging with the ability to detect hemoglobin, lipids, water and other light-absorbing chromophores, but with greater penetration depth than purely optical imaging modalities. As well as visualizing anatomical structures such as the micro-vasculature, it can also provide functional information in the form of blood oxygenation, blood flow and temperature. The tissue of interest is illuminated by a sufficiently short pulse of light from the photoacoustic probe. This light is absorbed by specific components within the tissue, such as hemoglobin or lipids, generating a mechanical wave whose frequency is in the ultrasound range. These signals can be detected by an ultrasound sensor, or array of ultrasound sensors, and the signals can be used to form an image of the tissue of interest with an image reconstruction algorithm.

The probe 7106 is one example of a photoacoustic probe, which includes a light source 7108, which can include a laser light. The probe 7106 includes an ultrasound transducer array (e.g., a piezoelectric element, or a Fabry-Perot Interferometer element) 7110 for detecting the optoacoustic or acoustic waves emanating from the tissue when illuminated by the light from the photoacoustic probe. The laser light can include a pulsed source in certain implementations. In some implementations, the light source is a light emitting diode. Sensor data from the transducer array 7110 is received by the processor 380 of controller 130 to generate a tissue image for characterization of tissue in order to generate alerts and control the delivery of the SSO₂ blood to the patient. The magnitude of the ultrasonic emission (i.e. the photoacoustic signal), which is proportional to the local energy deposition, reveals physiologically specific optical absorption contrast. For example, optical absorption is different for oxygenated hemoglobin vs deoxygenated hemoglobin, and this contrast is visible in the generated image. An image of the tissue area may show dark zones which represent ischemia in tissue, but as oxygenated blood flows into the ischemic tissue and O₂ diffuses through the tissue, the image will begin to light up. Two-dimensional or three-dimensional images of the targeted tissue area may be formed, wherein the image contrasts are indicative of the presence or change in the level of oxygen in the targeted tissue area. In certain implementations, an image of the targeted tissue area may be tracked over time to determine a change in oxygenation in the tissue area over time. For example, slices of the images may be taken and tracked over time. The expectation would be to see increased oxygen in the tissue over time as the SSO₂ therapy is delivered to the patient.

The processor 380 may generate a tomographic image of the tissue area. In certain implementations, a traditional ultrasonic image may be generated providing an image of the anatomical structure in the tissue area, including blood vessels and infarct. A photoacoustic image may also be generated to provide an image of oxygenation in the tissue area. The photoacoustic image can be overlaid on the ultrasonic image to see changes in infarct size in the tissue area. The system may include a separate photoacoustic probe and ultrasound receiver, or the two components may be part of single device. In certain implementations, a handheld ultrasound device may be coupled to the system, but modified to only receive acoustic waves, and to generate an image based on acoustic waves received from the photoacoustic-imaging probe. Any resulting photoacoustic image may be displayed on the user interface of the system or on remote or separate monitor or tablet. The image can be transmitted to the remote monitor or tablet wirelessly or via a wired connection.

In certain implementations, the light source may be a fiberoptic cable coupled to the gas enriched blood delivery catheter, positioning the light source closer to the targeted tissue area. The system may include hardware to pulse or tune the light or laser light.

In SSO₂ therapy, the infusion point for delivery of the oxygen enriched blood is often removed from the targeted tissue area or ischemic area (e.g., upstream by several mm from a vessel blockage). A system with photoacoustic imaging probe as described herein, through tuning and/or through different source hardware, may provide varying levels of imaging depth and tissue penetration, which allows for a more localized delivery of light, which better targets the tissue area of interest and improves acoustic wave generation and resulting imaging of the tissue area. The imaging depth may vary depending on the target tissue or may be selected depending on the distance of the light source from the targeted tissue area, for instance, the imaging depth might be 1-3 mm for blood vessels and 15-35 mm for the ventricular myocardium and 3-10 mm for atrial tissue. Exemplary imaging depths for various tissue targets using exemplary known photoacoustic (PAI) or optoacoustic (OAI) imaging platforms are provided in the below Table 1 (Schellenberg, Photoacoustics 11 (2018) 14-17).

TABLE 1
(continued)

| Subject | Target Tissue | Method | Resolution | Max Depth | | Speed | | SNR | Sensitivity |
|---|---|---|---|---|---|---|---|---|---|
| Human | Vascular structure | 2D OACT | A: 250 μm | 5-20 | mm | 0.1 | s | DNR | DNR |
| | in thyroid | (MSOT) | L: 730 μm | below | | | | | |
| | | | | surface | | | | | |
| | Radial artery | 2D OACT | A, L: 170 μm | DNR | | 0.02 | s | DNR | DNR |
| | | (MSOT) | | | | | | | |
| | Deep vasculature in | 3D OACT | A, L: 200 μm | DNR | | 0.1 | s | DNR | DNR |
| | hand and forearm | 3D OACT | A, L: 200 μm | 15 | mm | 0.1 | s | DNR | DNR |
| | Vasculature in palm | 3D OACT | A, L: 200 μm | 6 | mm | DNR | | DNR | DNR |
| | | (MSOT) | | | | | | | |
| | Hemoglobin and melanin | 3D OACT | A, L: 200 μm | 22 | mm | 0.1 | s | 5 [symbol] | DNR |
| | in breast tissue | (MSOT) | | | | | | | |
| | Hemoglobin, melanin, and | 3D OACT | A, L: 75 μm | 4.7 | mm | 0.05 | s | DNR | DNR |
| | lipid structures around | (MSOT) | | | | | | | |
| | hair follicles | | | | | | | | |
| | Hemoglobin and melanin | 2D OACT | A, L: 150 μm | DNR | | DNR | | DNR | DNR |
| | in non-melanoma skin | (MSOT) | | | | | | | |
| | cancer lesions | 3D OACT | A, L: 80 μm | DNR | | DNR | | DNR | DNR |
| | | (MSOT) | | | | | | | |
| | Hemoglobin in breast | 2D OACT | A, L: 250 μm | 30 | mm | 0.2 | s | DNR | DNR |
| | tissue | (MSOT) | | | | | | | |
| | | 2D OACT | DNR | 20 | mm | DNR | | DNR | DNR |
| | | (PAI) | | | | | | | |
| | Hemoglobin, lipids, and | 2D OACT | A, L: 115 μm | DNR | | 0.5 | s | DNR | DNR |
| | water content in breast | (MSOT) | | | | | | | |
| | tissue | | | | | | | | |
| | Radial artery and vein | 2D OACT | A: 86 μm | DNR | | 0.1 | s | DNR | DNR |
| | | (PAT) | L: 119 μm | | | | | | |
| | Melanoma tumor | 2D OACT | A: 86 μm | 10 | mm | DNR | | DNR | DNR |
| | | (PAT) | L: 119 μm | | | | | | |
| | Mole on finger | 3D OR-OMI | A: 30 μm | DNR | | 20 | s | DNR | DNR |
| | | (OR-PAM) | L: 12 μm | | | | | | |
| | Hemoglobin in | OACT | DNR | DNR | | DNR | | DNR | DNR |
| | intestinal walls | (MSOT) | | | | | | | |
| | Vasculature in prostate | 2D OACT | DNR | 15 | mm | 0.2 | s | DNR | DNR |
| | | (PAI) | | | | | | | |
| | Sentinel lymph nodes | 2D OACT | A, L: 300 μm | 50 | mm [symbol] | 1.33 | s | DNR | 400 cells/μL [symbol] |
| | in melanoma patients | (MSOT) | | | | | | | |
| | Sentinel lymph nodes | 2D OACT | DNR | DMR | | 0.2 | s | DNR | DNR |
| | tagged with dye | (PAT) | | | | | | | |
| | Arteries and veins | OACT | A, L: 115 μm | 8 | mm | 0.02 | s | 20 dB [symbol] | DNR |
| | in wrist | (MSOT) | | | | | | | |
| | Red mole on leg | 3D OR-DMI | A: 26 μm | 0.54 | mm [symbol] | 0.5 | s | 20 dB [symbol] | DNR |
| | | (OR-PAM) | L: 5 μm | | | | | | |
| | Vasculature in cuticle | 3D OR-DMI | A: 26 μm | 0.54 | mm [symbol] | 0.5 | s | 26 dB [symbol] | DNR |
| | | (OR-PAM) | L: 5 μm | | | | | | |
| | Vasculature in patients | 3D OMe | A: 4.5 μm | 1.5 | mm | DNR | | DNR | DNR |
| | with psoriasis | (RSOM) | L: 18.4 μm | | | | | | |
| | Vasculature in [symbol] of | 3D OMe | A: 8 μm | 1.5 | mm | DNR | | DNR | DNR |
| | patients with systematic | (RSOM) | L: 30 μm | | | | | | |
| | sclerosis | | | | | | | | |
| | Vasculature in foot | 2D OACT | A, L: 100 μm | 10 | mm | 0.1 | s | 14.3 [symbol] | DNR |
| | | (MSOT) | | | | | | | |

[symbol]

indicates data missing or illegible when filed

In certain implementations, the imaging range for myocardial tissue may depend on the placement of the source and on the size and layers of fat between the probe and the heart. When the light source and ultrasound transducer are external to the patient, the ultrasound can be tuned to receive signals from deeper tissues and higher powered pulsed lasers may be used to excite deeper target tissue. The initial response may be filtered such that the response from the upper layers is not received. When the light source is at the catheter site, e.g., a fiber optic cable in line with the catheter, which delivers light directly to the target tissue, the light source could be pulsed or continuous. This illumination could provide information regarding relaxation of the tissue after excitation, blood flow or oxygen diffusion. The source of signal would be targeted by the ultrasound externally.

In certain implementations, the photoacoustic image may be used to calculate blood flow. This may be accomplished by pulsing light, and looking at a response in the image over time, e.g., whether oxygenated blood is traveling or is static. This could indicate if there is lack of blood flow due to ischemia, or an increase of flow as SSO₂ therapy is effective. More blood flow means more oxygenation of the tissue and a reduction of ischemia.

The entire disclosures of U.S. Pat. Nos. 6,743,196, 6,582,387, 7,820,102 and 8,246,564 are expressly incorporated herein by reference.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device.

A computer program is a set of instructions that can be used, directly or indirectly, in a computer to perform some activity or bring about some result. A computer program can be written in any form of programming language, including compiled or interpreted languages, and it can be deployed in any form, including as a stand-alone program or as a module, component, subroutine, or other unit suitable for use in a computing environment. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks.

The computing device described herein may include, or be operatively coupled to communicate with, one or more mass storage devices for storing data files; such devices include magnetic disks, such as internal hard disks and removable disks; magneto-optical disks; and optical disks.

The terms “machine-readable medium,” “computer-readable medium,” and “processor-readable medium” as used herein, refer to any medium that participates in providing data that causes a machine to operate in a specific fashion. Using a computer system, various processor-readable media (e.g., a computer program product) might be involved in providing instructions/code to processor(s) for execution and/or might be used to store and/or carry such instructions/code (e.g., as signals).

In many implementations, a processor-readable medium is a physical and/or tangible storage medium. Such a medium may take many forms, including but not limited to, non-volatile media and volatile media. Non-volatile media include, for example, optical and/or magnetic disks. Volatile media include, without limitation, dynamic memory.

Common forms of physical and/or tangible processor-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, EPROM, a FLASH-EPROM, any other memory chip or cartridge, a carrier wave as described hereinafter, or any other medium from which a computer can read instructions and/or code.

Various forms of processor-readable media may be involved in carrying one or more sequences of one or more instructions to one or more processors for execution. Merely by way of example, the instructions may initially be carried on a flash device, a device including persistent memory, and/or a magnetic disk and/or optical disc of a remote computer. A remote computer might load the instructions into its dynamic memory and send the instructions as signals over a transmission medium to be received and/or executed by a computer system.

The computing device may be part of a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet. The computer system can include clients and servers. A client and server are generally remote from each other and typically interact through a network, such as the described one. The relationship of client and server arises by virtue of computer programs running on the respective computers and having a client-server relationship to each other.

Substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

Information and signals may be represented using any of a variety of different technologies and techniques. For example, data, instructions, commands, information, signals, and symbols that may be referenced throughout the above description may be represented by voltages, currents, electromagnetic waves, magnetic fields or particles, optical fields or particles, or any combination thereof.

The methods, systems, and devices discussed above are examples. Various alternative configurations may omit, substitute, or add various procedures or components as appropriate. Configurations may be described as a process which is depicted as a flow diagram or block diagram. Although each may describe the operations as a sequential process, many of the operations can be performed in parallel or concurrently. In addition, the order of the operations may be rearranged. A process may have additional stages not included in the figure. Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional stages or functions not included in the figure. Furthermore, examples of the methods may be implemented by hardware, software, firmware, middleware, microcode, hardware description languages, or any combination thereof. When implemented in software, firmware, middleware, or microcode, the program code or code segments to perform the tasks may be stored in a non-transitory processor-readable medium such as a storage medium. Processors may perform the described tasks.

Components, functional or otherwise, shown in the figures and/or discussed herein as being connected or communicating with each other are communicatively coupled. That is, they may be directly or indirectly connected to enable communication between them.

As used herein, including in the claims, “and” as used in a list of items prefaced by “at least one of” indicates a disjunctive list such that, for example, a list of “at least one of A, B, and C” means A or B or C or AB or AC or BC or ABC (i.e., A and B and C), or combinations with more than one feature (e.g., AA, AAB, ABBC, etc.). As used herein, including in the claims, unless otherwise stated, a statement that a function or operation is “based on” an item or condition means that the function or operation is based on the stated item or condition and may be based on one or more items and/or conditions in addition to the stated item or condition.

Having described several example configurations, various modifications, alternative constructions, and equivalents may be used without departing from the disclosure. For example, the above elements may be components of a larger system, wherein other rules may take precedence over or otherwise modify the application of the invention. Also, a number of operations may be undertaken before, during, or after the above elements are considered. Also, technology evolves and, thus, many of the elements are examples and do not bound the scope of the disclosure or claims. Accordingly, the above description does not bound the scope of the claims. Further, more than one invention may be disclosed.

Other embodiments are within the scope of the invention. For example, due to the nature of software, functions described above can be implemented using software, hardware, firmware, hardwiring, or combinations of any of these. Features implementing functions may also be physically located at various locations, including being distributed such that portions of functions are implemented at different physical locations.

The claims should not be read as limited to the described order or elements unless stated to that effect. It should be understood that various changes in form and detail may be made by one of ordinary skill in the art without departing from the spirit and scope of the appended claims. All implementations that come within the spirit and scope of the following claims and equivalents thereto are claimed.

What is claimed is:

1. A system for controlling gas enrichment therapy in a patient, the system comprising:
a gas enrichment system configured to enrich a liquid with gas to form a gas enriched liquid and to mix the gas enriched liquid with blood to form gas enriched blood;
a plurality of fluid conduits fluidly coupled to the gas enrichment system, at least one conduit of the plurality of fluid conduits configured for flow of the blood from the patient to the gas enrichment system, and at least one conduit of the plurality of conduits configured for flow of the gas-enriched blood from the gas enrichment system to the patient;
a blood pump coupled to at least one conduit of the plurality of fluid conduits, for pumping blood to and from the gas enrichment system and the patient;
at least one sensor configured to measure one or more blood oxygen parameters;
a user interface configured to receive user input and emit at least one of a visual alert and an audible alert; and a controller comprising:
a processor, a memory, and associated circuitry communicatively coupled to the at least one sensor and the user interface, wherein the processor is configured to:
receive one or more signals corresponding to a measured value of the one or more blood oxygen parameters from the at least one sensor, and
generate, based on the measured value, an alert through the user interface indicative of the measured value of the blood oxygen parameter, which is indicative of an effectiveness of the gas enrichment therapy.

2. The system according to claim 1, wherein the gas enrichment system is configured to enrich a liquid with oxygen to form an oxygen enriched liquid to be mixed with blood.

3. The system according to claim 1, wherein the one or more blood oxygen parameters comprises arterial pO₂.

4. The system according to claim 3, wherein the at least one sensor comprises a Clark electrode for measuring the pO₂.

5. The system according to claim 1, wherein the one or more blood oxygen parameters comprises arterial SO₂.

6. The system according to claim 3, wherein the processor compares the measured value for pO₂ to a preprogrammed target range for pO₂ of 760-1500 mmHg

7. The system according to claim 6, wherein the processor controls delivery of gas-enriched blood to the patient based on the comparison.

8. The system according to claim 1, wherein the one or more blood oxygen parameters is arterial SO₂ and the processor compares the measured value for SO₂ to an accepted normal range for arterial SO₂ is 90-100 percent.

9. The system according to claim 1, wherein the one or more blood oxygen parameters is arterial pO₂ and the processor compares the measured value for pO₂ to an accepted normal range for arterial pO₂ is 75-110 mmHg.

10. The system according to claim 1, wherein the gas-enrichment system comprises a cartridge.

11. The system according to claim 10, wherein the cartridge has three chambers.

12. A system for controlling gas enrichment therapy in a patient, the system comprising:
a gas enrichment system configured to enrich a fluid with gas to form a gas-enriched fluid and to mix the gas enriched fluid with blood to form gas enriched blood;
a plurality of fluid conduits fluidly coupled to the gas enrichment system, at least one conduit of the plurality of fluid conduits configured for flow of the blood from the patient to the gas enrichment system, and at least one conduit of the plurality of conduits configured for flow of the gas-enriched blood from the gas enrichment system to the patient;
a blood pump coupled to at least one conduit of the plurality of fluid conduits, for pumping blood to and from the gas enrichment system and the patient;
a catheter coupled to the conduit configured for flow of gas enriched blood to the patient, the catheter comprising one or more internal electrodes coupled thereto;
a plurality of external electrodes configured to be coupled to an external surface of a patient;
a user interface configured to receive user input and emit at least one of a visual alert and an audible alert; and
a controller comprising:
a processor, a memory, and associated circuitry communicatively coupled to the one or more internal electrodes of the catheter, the plurality of external electrodes and the user interface, wherein the processor is configured to:
receive a plurality of signals corresponding to measured impedance values from a tissue area between the one or more internal electrodes and plurality of external electrodes, and
generate an impedance tomographic map based at least in part on the measured impedance values, and
provide, through the user interface, information regarding blood perfusion in the tissue area based on the tomographic map, which information is indicative of an effectiveness of the gas enrichment therapy.

13. The system according to claim 12, wherein the gas enrichment system is configured to enrich a liquid with oxygen to form an oxygen enriched liquid to be mixed with blood.

14. The system according to claim 12, wherein the tissue perfusion information based on the tomographic map comprises increased blood perfusion and reduced infarct, which is represented by map zones having low impedance values.

15. The system according to claim 12, wherein the tissue area includes an infarct, and the processor is configured to compare the tomographic map of measured impedance values in the tissue area to a baseline tomographic map of measured impedance values in the tissue area to determine changes in blood perfusion or infarct size in the patient.

16. The system according to claim 12, wherein the processor is configured to tag map zones and analyze a change in tissue impedance for the tagged map zone over a period of time.

17. The system according to claim 16, wherein the processor is configured to calculate an average tissue impedance for the tagged map zone over a period of time.

18. The system according to claim 12, wherein the one or more catheter electrodes comprises a bipolar ecg electrode.

19. The system according to claim 12, wherein the processor is configured to cause the gas enrichment system to increase a level of O₂ saturation in the blood based on the tissue perfusion information.

20. The system according to claim 12, wherein the processor is configured to cause the pump to increase a flowrate of oxygen-enriched blood to the patient based on the tissue perfusion information.

21. The system according to claim 12, wherein the processor is configured to overlay the tomography map on an MRI or CT image of the tissue area showing an infarct zone, and the processor is configured to calculate the average impedance in the infarct zone.

22. A system for controlling gas enrichment therapy in a patient, the system comprising:
a gas enrichment system configured to enrich a fluid with gas to form a gas-enriched fluid and to mix the gas enriched fluid with blood to form gas enriched blood,
a plurality of fluid conduits fluidly coupled to the gas enrichment system, at least one conduit of the plurality of fluid conduits configured for flow of the blood from the patient to the gas enrichment system, and at least one conduit of the plurality of conduits configured for flow of the gas-enriched blood from the gas enrichment system to the patient,
a blood pump coupled to at least one conduit of the plurality of fluid conduits, for pumping blood to and from the gas enrichment system and the patient;
a nuclear magnetic resonance probe configured to measure a resonance signal of a target molecule in a target tissue;
a user interface configured to receive user input and emit at least one of a visual alert and an audible alert; and
a controller comprising:
a processor, a memory, and associated circuitry communicatively coupled to the magnetic resonance imaging probe and the user interface, wherein the processor is configured to:
receive one or more signals corresponding to a level of the target molecule in the target tissue based on the measured resonance signal of the molecule from the nuclear magnetic resonance imaging probe, and
generate, based on the measured value, an alert through the user interface indicating the level of the target molecule in the target tissue, which is indicative of an effectiveness of the gas enrichment therapy.

23. The system according to claim 22, wherein the gas enrichment system is configured to enrich a liquid with oxygen to form an oxygen enriched liquid to be mixed with blood.

24. The system according to claim 22, wherein the target molecule in the target tissue comprises oxygen in blood

25. The system according to claim 24, wherein the level of oxygen in blood refers to SO₂ in blood.

26. The system according to claim 22, wherein the target molecule in the target tissue comprises high-energy phosphate in blood, wherein a level of high-energy phosphate in blood is indicative of the tissue's metabolic state.

27. The system according to claim 22, wherein the processor is configured to generate a magnetic resonance image of the target tissue and analyze the image to detect the presence of the target molecule in the target tissue.

28. The system according to claim 22, wherein the magnetic resonance imaging probe comprises a magnetic coil wound around a peripheral portion of the catheter, the catheter coupled to the at least one conduit configured for flow of gas-enriched blood to the patient.

29. The system according to claim 22, wherein the magnetic resonance imaging probe comprises a magnetic resonance imaging receiver on the end of a catheter, the catheter coupled to the at least one conduit configured for flow of gas-enriched blood to the patient

30. A system for controlling supersaturated oxygen therapy in a patient, the system comprising:
a gas enrichment system configured to enrich a fluid with oxygen to form an oxygen enriched fluid and to mix the oxygen enriched fluid with blood to form oxygen enriched blood,
a plurality of fluid conduits fluidly coupled to the gas enrichment system, at least one conduit of the plurality of fluid conduits configured for flow of the blood from the patient to the gas enrichment system, and at least one conduit of the plurality of conduits configured for flow of the oxygen-enriched blood from the gas enrichment system to the patient,
a blood pump coupled to at least one conduit of the plurality of fluid conduits, for pumping blood to and from the gas enrichment system and the patient;
an O₂ fluorescence probe comprising one or more sensor molecules;
a user interface configured to receive user input and emit at least one of a visual alert and an audible alert; and
a controller comprising:
a processor, a memory, and associated circuitry communicatively coupled to the O₂ fluorescence probe and the user interface, wherein the processor is configured to:
receive one or more signals corresponding to a measured fluorescence of the sensor molecule on the O₂ fluorescence probe,
determine SO₂ in blood based on the one or more signals,
generate, based on the determined SO₂, an alert through the user interface indicating an effectiveness of the supersaturated oxygen therapy.

31. The system according to claim 30, wherein the O₂ fluorescence probe comprises a catheter.

32. The system according to claim 30, wherein the O₂ fluorescence probe comprises a sensor molecule coated onto an end of a fiber optic cable.

33. The system according to claim 30, wherein the sensor molecule comprises a fluorophore or phosphor.

34. The system according to claim 33, wherein the processor is configured to measure fluorescence signal decay from the sensor molecule due to quenching by O₂, wherein the signal decay time is proportional to SO₂ or pO₂ in the blood.

35. A system for controlling supersaturated oxygen therapy in a patient, the system comprising:
a gas enrichment system configured to enrich a fluid with oxygen to form an oxygen enriched fluid,
a pump,
a plurality of fluid conduits fluidly coupled to the pump, at least one conduit in the plurality of conduits configured for flow of oxygen-enriched fluid generated by the gas enrichment system into a patient's blood vessel;
a transcutaneous pO₂ probe configured to measure pO₂ in a tissue area;
a user interface configured to receive user input and emit at least one of a visual alert and an audible alert; and
a controller comprising:
a processor, a memory, and associated circuitry communicatively coupled to the transcutaneous pO₂ probe and the user interface, wherein the processor is configured to:
receive one or more signals corresponding to a measured value of the pO₂ in the tissue area from the transcutaneous pO₂ probe, and
generate, based on the measured value, an alert through the user interface indicating an effectiveness of the supersaturated oxygen therapy.

36. The system according to claim 35, wherein the at least one conduit comprises a catheter configured to inject oxygen-enriched saline into the patient's blood vessel.

37. The system according to claim 35, wherein the processor controls the delivery of the oxygen-enriched saline into the blood based on the measured pO₂ value.

38. The system according to claim 35, wherein the measured value of the pO₂ in the tissue area comprises pO₂ in myocardial tissue.

39. The system according to claim 35, wherein the measured value of the pO₂ in the tissue area comprises pO₂ in a coronary blood vessel.

40. A system for controlling supersaturated oxygen therapy in a patient, the system comprising:
a gas enrichment system configured to enrich a fluid with gas to form a gas-enriched fluid and to mix the gas enriched fluid with blood to form gas enriched blood,
a plurality of fluid conduits fluidly coupled to the gas enrichment system, at least one conduit of the plurality of fluid conduits configured for flow of the blood from the patient to the gas enrichment system, and at least one conduit of the plurality of conduits configured for flow of the gas-enriched blood from the gas enrichment system to the patient,
a blood pump coupled to at least one conduit of the plurality of fluid conduits, for pumping blood to and from the gas enrichment system and the patient;
a photoacoustic imaging light source configured to illuminate a tissue area with a pulse of light;
an ultrasonic sensor configured to detect acoustic waves generated by light-absorbing components in the tissue area responsive to illumination by the pulse of light;
a user interface configured to receive user input and emit at least one of a visual alert and an audible alert; and
a controller comprising:
a processor, a memory, and associated circuitry communicatively coupled to the photoacoustic imaging probe, the ultrasonic sensor and the user interface, wherein the processor is configured to:
receive one or more signals corresponding to the detected acoustic waves,
generate, based on the detected acoustic waves, an image, and
provide, through the user interface, blood oxygenation information about the tissue area based on the image, which information is indicative of an effectiveness of the supersaturated oxygen therapy.

41. The system according to claim 40, wherein the gas enrichment system is configured to enrich a liquid with oxygen to form an oxygen enriched liquid to be mixed with blood.

42. The system according to claim 40, wherein the processor controls the delivery of oxygen-enriched blood to the patient based on tissue or blood oxygenation information from the image

43. The system according to claim 40, wherein the image is tracked over time to determine a change in blood oxygenation in the tissue area over time.

44. The system according to claim 40, wherein the image is tracked over time to determine a presence of or change in blood flow or blood oxygenation in the tissue area over time.

45. The system according to claim 40, wherein the photoacoustic imaging light source comprises a fiberoptic cable coupled to a catheter, the catheter configured to deliver the gas-enriched blood to the patient.

46. The system according to claim 40, wherein the processor is further configured to generate a tomographic image of the tissue area.

47. The system according to claim 40, wherein the photoacoustic imaging light source comprises a laser or pulsed laser diode for generating the pulse of light.

48. The system according to claim 40, wherein the blood oxygenation information comprises a change in oxygenated hemoglobin levels represented by a contrast in the image that results from optical absorption properties differing for oxygenated hemoglobin and deoxygenated hemoglobin.

49. The system according to claim 40, wherein the photoacoustic imaging light source comprises a light emitting diode for generating the pulse of light.

50. The system according to claim 40, wherein the ultrasonic sensor comprises a piezoelectric element.

51. The system according to claim 50, wherein the piezoelectric element comprises a linear, piezoelectric, ultrasound transducer array.

52. The system according to claim 40, wherein the ultrasonic sensor comprises a Fabry-Perot Interferometer (FPI) element.

53. The system according to claim 52, wherein the processor is further configured to raster scan the FPI.

54. The system according to claim 40, wherein the pulse of light is in a visible portion of an electromagnetic spectrum.

55. The system according to claim 40, wherein the pulse of light is within a near-infrared portion of an electromagnetic spectrum.

56. The system according to claim 40, wherein the processor is further configured to generate a two-dimensional image of the tissue area.

57. The system according to claim 40, wherein the processor is further configured to generate a three-dimensional image of the tissue area.

58. A system for controlling gas enrichment therapy in a patient, the system comprising:
a gas enrichment system configured to enrich a liquid with gas to form a gas enriched liquid and to mix the gas enriched liquid with arterial blood to form gas enriched blood;
a plurality of fluid conduits fluidly coupled to the gas enrichment system, at least one conduit of the plurality of fluid conduits configured for flow of the blood from the patient to the gas enrichment system, and at least one conduit of the plurality of conduits configured for flow of the gas-enriched blood from the gas enrichment system to the patient;
a blood pump coupled to at least one conduit of the plurality of fluid conduits, for pumping blood to and from the gas enrichment system and the patient;
at least one sensor configured to measure one or more physiological parameters;
a user interface configured to receive user input and emit at least one of a visual alert and an audible alert; and a controller comprising:
a processor, a memory, and associated circuitry communicatively coupled to the at least one sensor and the user interface, wherein the processor is configured to:
receive one or more signals corresponding to a measured value of the one or more physiological parameters from the at least one sensor, and
generate, based on the measured value, an alert through the user interface indicative of the measured value of physiological parameter, which is indicative of an effectiveness of the gas enrichment therapy.

59. The system according to claim 58, wherein the gas enrichment system is configured to enrich a liquid with oxygen to form an oxygen enriched liquid to be mixed with blood.

60. The system according to claim 58, wherein the one or more physiological parameters is a blood oxygen parameter, which comprises arterial pO₂.

61. The system according to claim 60, wherein the at least one sensor comprises a Clark electrode for measuring the pO₂ in blood.

62. The system according to claim 58, wherein the one or more physiological parameters is a blood oxygen parameter, which comprises arterial SO₂.

63. The system according to claim 60, wherein the processor compares the measured value for pO₂ to a preprogrammed target range for pO₂ of 760-1500 mmHg

64. The system according to claim 63, wherein the processor controls delivery of gas-enriched blood to the patient based on the comparison.

65. The system according to claim 62, wherein the processor compares the measured value for SO₂ to an accepted normal range for arterial SO₂ of 90-100 percent.

66. The system according to claim 60, wherein the processor compares the measured value for pO₂ to an accepted normal range for arterial pO2, which is 75-110 mmHg.

67. The system according to claim 58, wherein the gas-enrichment system comprises a cartridge.

68. The system according to claim 67, wherein the cartridge has three chambers.

69. The system according to claim 58, wherein the physiological parameter is arterial blood pressure.

70. The system according to claim 58, wherein the physiological parameter is an electrical activity of the heart measured by an ECG sensor.

71. A method for controlling supersaturated oxygen therapy in a patient, the method comprising:
measuring, via one or more sensors, one or more blood oxygen parameters of the patient;
transmitting one or more signals to a processor, the one or more signals corresponding to a measured value of the one or more blood oxygen parameters from the at least one sensor; and
generating, based on the measured value, an alert through a user interface indicating a measured value of the blood oxygen parameter indicative of an effectiveness of the supersaturated oxygen therapy.

72. The method of claim 71, wherein measuring comprises measuring via a sensor positioned in a catheter.

73. The method of claim 71, wherein measuring comprises measuring pO₂ of the blood.

74. The method of claim 71, wherein measuring comprises measuring SO₂ of the blood.

75. The method of claim 71, further comprising comparing the measured value for the one or more blood oxygen parameters to an accepted normal range for the one or more blood oxygen parameters in non-ischemic tissue.

76. The method of claim 75, further comprising controlling, via the processor, delivery of gas-enriched blood to the patient based on the comparison of the measured value to the accepted normal range.

77. A method for controlling gas enrichment therapy in a patient, the method comprising:
measuring, impedance values from a tissue area between one or more internal catheter electrodes and plurality of external electrodes;
generating a tomographic map of the measured impedance values, and
providing, through a user interface, tissue perfusion information regarding blood perfusion in the tissue area based on the tomographic map, which information is indicative of an effectiveness of the gas enrichment therapy.

78. The method according to claim 77, further comprising tagging map zones and analyzing a change in tissue impedance for the tagged map zone over a period of time.

79. The method according to claim 78, further comprising calculating an average tissue impedance for the tagged map zone over a period of time.

80. The method according to claim 77, further comprising causing a gas enrichment system to increase a level of O₂ saturation in the blood based on the tissue perfusion information.

81. The method according to claim 77, further comprising causing a pump to increase a flowrate of oxygen-enriched blood to the patient based on the tissue perfusion information.

82. The method according to claim 77, further comprising overlaying the tomography map on an MRI or CT image of the tissue area showing an infarct zone, and calculating the average impedance in the infarct zone.

83. A method for controlling gas enrichment therapy in a patient, the method comprising:
measuring one or more tissue parameters of a resonance of a target molecule in a target tissue using a nuclear magnetic resonance probe;
receiving one or more signals corresponding to a level of a target molecule in a target tissue based on the measured resonance of the molecules from a nuclear magnetic resonance imaging probe, and
generating, based on the measured value, an alert through the user interface, the alert indicating the level of the target molecule in the target tissue, which is indicative of an effectiveness of the gas enrichment therapy.

84. The method according to claim 83, further comprising generating a magnetic resonance image of the target tissue and analyze the image to detect the presence of the target molecule in the target tissue.

85. A method for controlling supersaturated oxygen therapy in a patient, the method comprising:
measuring fluorescence of a sensor molecule on an O₂ fluorescence probe;
receiving one or more signals corresponding to the measured fluorescence of the sensor molecule on the O₂ fluorescence probe;
determining SO₂ in blood based on the one or more signals; and
generating, based on the determined SO₂, an alert through the user interface indicating an effectiveness of the supersaturated oxygen therapy.

86. The method according to claim 85, further comprising measuring fluorescence signal decay from the sensor molecule due to quenching by O₂, wherein the signal decay time is proportional to SO₂ in the blood.

87. A method for controlling supersaturated oxygen therapy in a patient, the method comprising:
measuring pO₂ in a tissue area using a transcutaneous pO₂ probe;
receiving one or more signals corresponding to the measured pO₂ in the tissue are from the transcutaneous pO₂ probe;
generating, based on the measured pO₂, an alert through the user interface indicating an effectiveness of the supersaturated oxygen therapy.

88. The method according to claim 87, further comprising controlling a delivery of oxygen-enriched saline into blood based on the measured pO₂ value.

89. A method for controlling supersaturated oxygen therapy in a patient, the method comprising:
illuminating a tissue area with a pulse of light from a photoacoustic imaging light source;
detecting acoustic waves generated by light-absorbing components in the tissue area responsive to illumination by the pulse of light;
generating, based on the detected acoustic waves, an image; and
providing, through a user interface, blood oxygenation information about the tissue area based on the image, which information is indicative of an effectiveness of the supersaturated oxygen therapy.

90. The method according to claim 89, further comprising controlling delivery of oxygen-enriched blood to the patient based on blood oxygenation information from the image

91. The method according to claim 89, further comprising, further comprising tracking the image over time to determine a change in blood oxygenation in the tissue area over time.

92. The method according to claim 89, further comprising generating a tomographic image of the tissue area.

93. The method according to claim 89, further comprising generating a two-dimensional image of the tissue area.

94. The method according to claim 89, further comprising generating a three-dimensional image of the tissue area.

95. A method for controlling gas enrichment therapy in a patient, the method comprising:
measuring, via one or more sensors, one or more physiological parameters of the patient;
transmitting one or more signals to a processor, the one or more signals corresponding to a measured value of the one or more physiological parameters from the at least one sensor; and
generating, based on the measured value, an alert through a user interface indicating a measured value of the physiological parameter indicative of an effectiveness of the gas enrichment therapy.

96. The system of claim 1, wherein the gas enriched liquid comprises a supersaturated oxygen liquid.

97. The system of claim 96, wherein the supersaturated oxygen liquid has an O₂ concentration of 0.1-6 ml O2/ml liquid (STP).

98. The system of claim 96 or 1, wherein the gas-enriched blood comprises a supersaturated oxygen enriched blood.

99. The system of claim 98, wherein the supersaturated oxygen enriched blood has a pO₂ of 600-1500 mmHg.

100. The system of claim 12, wherein the gas enriched fluid comprises a supersaturated oxygen liquid.

101. The system of claim 100, wherein the supersaturated oxygen liquid has an O₂ concentration of 0.1-6 ml O₂/ml liquid (STP).

102. The system of claim 12, wherein the gas-enriched blood comprises a supersaturated oxygen enriched blood.

103. The system of claim 102, wherein the supersaturated oxygen enriched blood has a pO₂ of 600-1500 mmHg.

104. The system of claim 30, wherein the oxygen enriched fluid comprises a supersaturated oxygen liquid.

105. The system of claim 104, wherein the supersaturated oxygen liquid has an O₂ concentration of 0.1-6 ml O₂/ml liquid (STP).

106. The system of claim 30, wherein the oxygen-enriched blood comprises a supersaturated oxygen enriched blood.

107. The system of claim 106, wherein the supersaturated oxygen enriched blood has a pO₂ of 600-1500 mmHg.

108. The system of claim 35, wherein the oxygen enriched fluid comprises a supersaturated oxygen liquid.

109. The system of claim 108, wherein the supersaturated oxygen liquid has an O₂ concentration of 0.1-6 ml O₂/ml liquid (STP).

110. The system of claim 35, wherein the oxygen-enriched blood comprises a supersaturated oxygen enriched blood.

111. The system of claim 110, wherein the supersaturated oxygen enriched blood has a pO2 of 600-1500 mmHg.

112. The system of claim 40, wherein the gas enriched fluid comprises a supersaturated oxygen liquid.

113. The system of claim 112, wherein the supersaturated oxygen liquid has an O₂ concentration of 0.1-6 ml O₂/ml liquid (STP).

114. The system of claim 40, wherein the gas-enriched blood comprises a supersaturated oxygen enriched blood.

115. The system of claim 114, wherein the supersaturated oxygen enriched blood has a pO₂ of 600-1500 mmHg.

116. The system of claim 58, wherein the gas enriched liquid comprises a supersaturated oxygen liquid.

117. The system of claim 116, wherein the supersaturated oxygen liquid has an O₂ concentration of 0.1-6 ml O₂/ml liquid (STP).

118. The system of claim 58, wherein the gas-enriched blood comprises a supersaturated oxygen enriched blood.

119. The system of claim 118, wherein the supersaturated oxygen enriched blood has a pO₂ of 600-1500 mmHg.

120. The system of claim 58, wherein the gas enrichment therapy is a supersaturated oxygen therapy.
